(19) United States
(12) Patent Application Publication
(10) Pub. No.: US 2014/0039480 A1
(43) Pub. Date: Feb. 6, 2014

(54) BIPOLAR ENDOSCOPIC TISSUE ABLATOR WITH SIMPLE CONSTRUCTION

(71) Applicant: ELECTROMEDICAL ASSOCIATES LLC, Bethesda, MD (US)

(72) Inventor: Robert A. Van Wyk, St. Pete Beach, FL (US)

(73) Assignee: ElectroMedical Associates LLC, Bethesda, MD (US)

(21) Appl. No.: 13/762,666

(22) Filed: Feb. 8, 2013

Related U.S. Application Data
(60) Provisional application No. 61/742,270, filed on Aug. 6, 2012.

(51) Int. Cl. A61B 18/14; A61B 18/18

(52) U.S. Cl. CPC A61B 18/14; A61B 18/18
USPC 606/33; 606/41

(57) ABSTRACT

The present invention relates generally to the field of electrosurgery, more particularly to electrosurgical devices and methods that employ high frequency voltage to cut, ablate and/or coagulate tissue in conductive fluid and semi-dry environments, even more particularly to ablation electrodes designed for the bulk removal of tissue by vaporization as opposed to the simple cutting of tissue or coagulation of bleeding vessels. Further to a need in the art, the present invention provides bipolar ablators of simple construction.

### PRIORITY

This application claims the benefit of U.S. Provisional Application Ser. No. 61/742,270 filed Aug. 6, 2012, the entire contents of which are incorporated by reference herein.

### FIELD OF THE INVENTION

The present invention relates generally to the field of electrosurgery and, more particularly, to electrosurgical devices and methods that employ high frequency voltage to cut, ablate and/or coagulate tissue in conductive fluid and semi-dry environments.

### BACKGROUND OF THE INVENTION

Due to their ability to accomplish outcomes with reduced patient pain and accelerated return of the patient to normal activities, least invasive surgical techniques have gained significant popularity. Arthroscopic surgery in particular, wherein the intra-articular space is filled with fluid, allows orthopedic surgeons to efficiently perform procedures using special purpose instruments designed specifically for arthroscopists. Among these special purpose tools are various manual graspers and biters, powered shaver blades and burs, and electrosurgical devices. The last several years has seen the development of specialized arthroscopic electrosurgical electrodes called ablators. Examples of such instruments include ArthroWands manufactured by Arthrocare (Austin, Tex.), VAPR electrodes manufactured by Mitek Products Division of Johnson & Johnson (Raynham, Mass.), electrodes by Smith and Nephew, Inc. (Andover, Mass.), and OPES ablators by Arthrex, Inc. (Naples, Fla.). These ablator electrodes differ from conventional arthroscopic electrosurgical electrodes in that they are designed for the bulk removal of tissue by vaporization as opposed to the simple cutting of tissue or coagulation of bleeding vessels. While standard electrodes are capable of ablation, their geometries are generally inefficient in accomplishing this task. While the tissue removal rates of ablator electrodes are lower than those of shaver blades, ablators are used because they achieve hemostasis (stop bleeding) during use and thus are able to efficiently remove tissue from bony surfaces. Ablator electrodes are generally used in an environment filled with electrically conductive fluid.

Ablator electrodes are available in a variety of sizes and configurations to suit a variety of procedures. For example, ablators for use in ankle or elbow arthroscopy tend to be smaller than those used in the knee or shoulder. In each of these sizes, a variety of configurations are produced to facilitate access to various structures within the joint being treated. These configurations differ in the working length of the electrode (the maximum distance that an electrode can be inserted into a joint), in the size and shape of their ablating surfaces, and in the angle between the ablating face and the axis of the electrode shaft. Electrodes are typically designated by the angle between a normal to the ablating surface and the axis of the electrode shaft, and by the size of their ablating surface and any associated insulator.

Primary considerations of surgeons when choosing a particular configuration of ablator for a specific procedure are (a) its convenience of use (i.e., its ease of access to certain structures) and (b) the speed with which the ablator will be able to complete the required tasks. When choosing between two configurations capable of accomplishing a particular task, surgeons will generally choose the ablator with the larger ablating surface to remove tissue more quickly. This is particularly true for procedures during which large volumes of tissue must be removed. One such procedure is acromioplasty or the reshaping of the acromion, a bony continuation of the scapular spine that hooks over anteriorly and articulates with the clavicle (collar bone) to form the acromioclavicular joint.

The underside of the acromion is covered with highly vascular tissue that tends to bleed profusely when removed with a conventional powered cutting instrument such as an arthroscopic shaver blade. Ablator electrodes are used extensively during this procedure since they are able to remove tissue without the bleeding. Ablation in the area under the acromion is most efficiently accomplished using an electrode on which a line normal to the ablating surface is perpendicular to the axis of the ablator shaft. Such an electrode is designated as a “90 Degree Ablator” or a “side effect” ablator. Exemplary of such electrodes are the “3.2 mm 90 Degree Three-Rib UltrAblator” by Linvatec Corporation (Largo, Fla.), the “90 Degree Ablator” and “90 Degree High Profile Ablator” by Oratec Interventions, the “Side Effect VAPR Electrode” by Mitek Products Division of Johnson and Johnson, and the “3.5 mm 90 Degree Arthrowand,” “3.6 mm 90 Degree Lo Pro Arthrowand,” and “4.5 mm 90 Degree Eliminator Arthrowand” by Arthrocare Corporation.

The above-mentioned 90-degree ablator electrodes may be divided into two categories: (i) electrodes of simple construction, wherein RF energy is conducted to the ablator tip by an insulated metallic rod or tube; and (ii) electrodes of complex construction, which use wires to conduct power to the tip.

Ablator electrodes having a simple geometry are produced by Linvatec Corporation (as described in U.S. Pat. No. 6,149,646) and Arthrex, Inc. and are monopolar instruments, that is, the circuit to the electrosurgical generator is completed by means of a dispersive pad (also called a return pad) placed on the patient at a distance from the surgical site. The distal end of the ablator rod is provided with a suitable geometry, either ribbed or annular, and the distal tip of the rod is bent to a predetermined angle to the axis of the rod. For a 90-degree electrode, this predetermined angle is 90 degrees. The rod diameter may be locally reduced in the region near its distal tip to reduce the radius of the bend. The rod is insulated up to the ablation face on the rod distal tip using polymeric insulation. A ceramic insulator may be added to prevent charring of the polymeric insulation.

Ninety-degree ablator electrodes having a complex construction, such as those in which the active electrode is attached to the electrosurgical generator via cables passing through an elongated tubular member, are produced by Arthrocare Corporation (U.S. Pat. No. 5,944,646 and others) and the Mitek Products Division of Johnson & Johnson. Typically, these electrodes are bipolar instruments, having a return electrode on the instrument in close proximity to the active electrode. Bipolar arthroscopy electrodes of this type are constructed of a tubular member upon which one or more electrodes (herein referred to as active electrodes) are mounted and connected via one or more cables to an electrosurgical generator, the leads passing through the lumen of the tubular member. The active electrodes are isolated electrically from the tubular member and rigidly mounted to the tubular member by a ceramic insulator affixed to the tubular member. The tubular member is electrically isolated from the conductive fluid medium by a polymeric coating, except for an area at the distal tip in the vicinity of the active electrode. The proximal end of the tubular member is connected electrically to the electrosurgical generator via one or more cables. During use, current flows from the active electrode through the conductive fluid medium to the uninsulated portion of the tubular member, which functions as a return electrode in close proximity to the active electrode. These ablators may be equipped with aspiration, that is, with a means for connecting the ablation device to an external vacuum source such that bubbles and debris produced by the tissue vaporization are removed from the ablation site. If these ablators of complex construction are equipped with aspiration, the portion of the aspiration pathway that lies within the tubular member is formed from a small-diameter polymeric tube.

Van Wyk in U.S. Pat. No. 6,840,937 teaches a monopolar arthroscopy ablator of simple construction. There is a need for a bipolar ablator of simple construction with its associated benefits of simplicity of construction and reduced costs. The present invention meets this need.

### SUMMARY OF THE INVENTION

It is accordingly an object of this invention to produce a bipolar ablator wherein the electrical energy is conducted to the active electrode by a rigid structural member rather than wires.

It is also an object of this invention to produce a bipolar ablator wherein the rigid structural member conducting electrical energy to the active electrode is tubular so as to also provide an aspiration path through a lumen within it.

It is also an objective of this invention to produce a bipolar ablator of high efficiency which may be connected to a general purpose radio frequency generator.

It is further an objective of this invention to produce a safe bipolar ablator of high reliability due to efficient heat removal from the active region by the structural conductive members.

It is further an objective of this invention to produce a low cost bipolar ablator with either finger control or foot control.

It is further an objective of this invention to produce a bipolar ablator that is compatible with multiple types of general-purpose generator, thus avoiding possible scheduling conflicts associated with dedicated generators. These and other objects are accomplished in the invention herein disclosed, directed to an endoscopic bipolar ablator of simple construction. That is, an endoscopic ablator in which energy is conducted to the active electrode by a structural member (rather than wires) and in which the return electrode is on the device in moderate proximity to the active electrode. It will be understood by those skilled in the art that one or more aspects of this invention can meet certain of the above objectives, while one or more other aspects can meet certain other objectives. Each objective may not apply equally, in all its respects, to every aspect of this invention. As such, the preceding and foregoing objects should be viewed in the alternative with respect to any one aspect of this invention.

Ablators formed in accordance with the principles of this invention have a proximal handle portion and an elongate distal portion designed to be inserted into the joint space during use. The device is connected to an electrosurgical generator by a cable passing from the proximal end of the handle portion, the cable having connection means for both the “output” and “return” connectors of the generator. When used with a special purpose generator, i.e., one designed specifically for use with the subject probe or similar, the active and return connections may use a single multi-pin connector. In a preferred embodiment, the ablator is connected to the “monopolar connectors” socket (also referred to as the receptacle) of a standard multi-purpose generator. The proximal portion of the cable is divided into two cable portions, a first portion having a connector for connecting to the monopolar output of the generator and a second portion having a connector for connecting to the generator monopolar return.

In a preferred embodiment, the handle has buttons on its top surface for controlling output of the electrosurgical generator, the buttons being analogous to those on a standard electrosurgical pencil (for instance, the ESP1 Disposable Pencil by Bovie Medical, Clearwater, Fla.). In a preferred embodiment, the proximal portion of the cable connecting the device to the generator output is configured like the standard connector for a hand-controlled electrosurgical pencil. This allows devices of this embodiment to connect to the “hand control” monopolar connectors of any standard multi-purpose generator and be controlled by buttons on the handle. In embodiments for use with a dedicated, special-purpose generator, connections for control of the generator by the buttons on the handle may be via pins within the multi-pin connector providing active and return paths to the device.

In still other embodiments, the device active and return energy paths may be connected to the bipolar output of a multi-purpose (also referred to as general purpose) generator, the generator being controlled by a foot-activated control.

In yet other embodiments, the device is connected to the monopolar “foot control” output of a general-purpose generator, and the return connected to the generator monopolar return. The device is then controlled by a foot activated control connected to the generator in the usual manner.

In yet further embodiments, the device may be provided with both a “return electrode” and one or more “floating electrodes” as described in U.S. Pat. No. 8,308,724 to Carmel et al. The separation between the active and floating electrodes is preferably between two and ten millimeters, and more preferably between three and six millimeters.

In a preferred embodiment, the structural member conducting energy to the active electrode is tubular, the lumen of the member serving as an aspiration path for removing gaseous and liquid ablation byproducts. Flow through the tubular member also serves to cool the device. However, non-aspirating devices embodiments are also contemplated. In such devices, the energy carrying structural member may be tubular or a solid rod, the rod cooling the active electrode by conduction of heat away from the ablating surface.

The above-noted objects and features of the invention will become more fully apparent when the following detailed description is read in conjunction with the accompanying figures and/or examples. However, it is to be understood that both the foregoing summary of the invention and the following detailed description are of preferred embodiments and not restrictive of the invention or other alternate embodiments of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art having knowledge of electrode design. Such objects, features, benefits and advantages apparent from the above in conjunction with the accompanying examples, data, figures and all reasonable inferences to be drawn there-from are specifically incorporated herein.

### BRIEF DESCRIPTION OF THE FIGURES

Various aspects and applications of the present invention will become apparent to the skilled artisan upon consideration of the brief description of the figures and the detailed description of the present invention and its preferred embodiments that follows:

FIG. 1 is a plan view of an insulator for a bipolar ablator formed in accordance with the principles of this invention.

FIG. 2 is a perspective view of the objects of FIG. 1.

FIG. 3 is a side elevational view of the objects of FIG. 1.

FIG. 4 is a distal axial view of the objects of FIG. 1.

FIG. 5 is a side elevational sectional view of the objects of FIG. 1 at location A-A of FIG. 1.

FIG. 6 is an auxiliary view of the objects of FIG. 1.

FIG. 7 is a perspective view of an active element for a bipolar ablator formed in accordance with the principles of this invention.

FIG. 8 is a plan view of the objects of FIG. 7.

FIG. 9 is a side elevational view of the objects of FIG. 7.

FIG. 10 is a side elevational sectional view of the objects of FIG. 7 at location A-A of FIG. 8.

FIG. 11 is a plan view of an assembly formed of the active element of FIG. 7 together with a metallic tubular element.

FIG. 12 is a side elevational view of the objects of FIG. 11.

FIG. 13 is an expanded distal axial view of the objects of FIG. 11.

FIG. 14 is a perspective view of the distal portion of the objects of FIG. 11.

FIG. 15 is a plan view of the objects of FIG. 14.

FIG. 16 is a side elevational view of the objects of FIG. 14.

FIG. 17 is a side elevational sectional view of the objects of FIG. 14 at location A-A of FIG. 11.

FIG. 18 is a plan view of an insulated inner assembly for a bipolar ablator formed in accordance with the principles of this invention.

FIG. 19 is a side elevational view of the objects of FIG. 18.

FIG. 20 is an expanded distal axial view of the objects of FIG. 18.

FIG. 21 is a plan view of the distal portion of the objects of FIG. 18.

FIG. 22 is a side elevational view of the objects of FIG. 21.

FIG. 23 is a perspective view of the objects of FIG. 21.

FIG. 24 is a side elevational sectional view of the objects of FIG. 21 at location A-A of FIG. 18.

FIG. 25 is a perspective view of a distal assembly for a bipolar ablator formed in accordance with the principles of this invention.

FIG. 26 is a plan view of the objects of FIG. 25.

FIG. 27 is a side elevational view of the objects of FIG. 25.

FIG. 28 is an expanded distal axial view of the objects of FIG. 25.

FIG. 29 is a plan view of the distal portion of the objects of FIG. 25.

FIG. 30 is a side elevational view of the objects of FIG. 29.

FIG. 31 is a perspective view of the objects of FIG. 29.

FIG. 32 is a side elevational sectional view of the objects of FIG. 32 at location A-A of FIG. 26.

FIG. 33 is a plan view of a bipolar ablator formed in accordance with the principles of this invention.

FIG. 34 is a side elevational view of the objects of FIG. 33.

FIG. 35 is an expanded distal axial view of the objects of FIG. 33.

FIG. 36 is a perspective view of the objects of FIG. 33.

FIG. 37 depicts a bipolar ablator system constructed in accordance with the principles of this invention.

FIG. 38 is a side elevational sectional view of the distal portion of a bipolar ablator formed in accordance with the principles of this invention during use showing the aspiration flow.

FIG. 39 is a side elevational sectional view of the distal portion of a bipolar ablator formed in accordance with the principles of this invention during use showing the current flow.

FIG. 40 is a plan view of an alternate embodiment of the invention.

FIG. 41 is a side elevational view of the objects of FIG. 40.

FIG. 42 is a perspective view of the objects of FIG. 40.

FIG. 43 is a plan view of the distal portion of the objects of FIG. 40.

FIG. 44 is a side elevational view of the objects of FIG. 43.

FIG. 45 is a perspective view of the objects of FIG. 43.

FIG. 46 is a side elevational sectional view of the objects of FIG. 43 at location A-A of FIG. 40.

FIG. 47 is a schematic view of an ablating system constructed in accordance with the principles of this invention comprising the alternate embodiment of FIG. 39

FIG. 48 is a plan view of the distal portion of an alternate embodiment constructed in accordance with the principles of this invention.

FIG. 49 is a side elevational view of the objects of FIG. 48.

FIG. 50 is a perspective view of the objects of FIG. 48.

FIG. 51 is a side elevational sectional view of the objects of FIG. 48 at location A-A of FIG. 48.

FIG. 52 is a distally-facing perspective view of an alternate embodiment formed in accordance with the principles of this invention.

FIG. 53 is a proximally facing perspective view of the objects of FIG. 52.

FIG. 54 is an expanded perspective view of the distal portion of the objects of FIG. 53.

FIG. 55 is a schematic view of an ablating system constructed in accordance with the principles of this invention comprising the alternate embodiment of FIG. 52.

FIG. 56 is a plan view of the distal portion of an alternate embodiment formed in accordance with the principles of this invention.

FIG. 57 is a side elevational view of the objects of FIG. 56.

FIG. 58 is a perspective view of the objects of FIG. 56.

FIG. 59 is a side elevational sectional view of the objects of FIG. 56 at location A-A of FIG. 56.

### DESCRIPTION OF THE PREFERRED EMBODIMENTS

The present invention constitutes an improvement in the field of electrosurgery, particularly an improvement to the cost and manufacture of bipolar devices that employ high frequency voltage to cut, ablate and/or coagulate tissue in conductive fluid and semi-dry environments, more particularly bipolar ablators designed for the bulk removal of tissue by vaporization as opposed to the simple cutting of tissue or coagulation of bleeding vessels.

Bipolar ablation devices constructed in accordance with the principles of this invention tend to be characterized by a proximal portion forming a handle and an elongate distal portion comprising concentric inner and outer rigid conductive elements insulated from each other by an insulating layer. The inner conductive element provides a conductive path to an active element (or active electrode or active electrode assembly) mounted to its distal end whereas the outer conductive element provides a path for a return electrode disposed at its distal end. The inner conductive element may be a solid rod to which an active electrode is mounted or, alternatively, may be an open tube that provides a path for the aspiration of ablation bubbles, irrigant and debris from the ablation site. The active electrode may be a single unitary element having proximal end configured for attachment to the distal end of the inner conductive element and a distal end forming an ablating surface. Alternatively, the active electrode may take the form of an assembly and thus be composed of a distal electrode element that terminates in an ablating surface and a proximal electrode element that provides a means for mounting to the distal end of the inner conductive element. In this manner, a current path and optionally an aspiration path is maintained between the distal electrode element and the inner conductive element. In preferred embodiments, the distal ablating surface is located off-axis from the longitudinal axis of the inner conductive element. In addition, the ablating surface may include one or more (or a plurality of) protuberances or cavities for the purpose of creating at least one region of increased current density.

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### Elements of the Present Invention

Unless otherwise defined, all technical and scientific terms used herein have the same meaning as commonly understood by one of ordinary skill in the art to which this invention belongs. In case of conflict, the present specification, including following definitions, will control.

The words “a”, “an”, and “the” as used herein mean “at least one” unless otherwise specifically indicated.

The term “proximal” refers to that end or portion which is situated closest to the user; in other words, the proximal end of a bipolar ablator of the instant invention will typically include the handle portion.

The term “distal” refers to that end or portion situated farthest away from the user; in other words, the distal end of a bipolar ablator of the instant invention will typically include the active element/active electrode portion.

In certain embodiments, the present invention makes reference to “fluid(s)”. As used herein, the term “fluid(s)” refers to liquid(s), either electrically conductive or non-conductive, and to gaseous material, or a combination of liquid(s) and gas(es). In the context of the present invention, the term “fluid” extends to body fluids, examples of which include, but not limited to, blood, peritoneal fluid, lymph fluid, pleural fluid, gastric fluid, bile, and urine.

The present invention makes reference to the ablation, coagulation and vaporization of tissue. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. The present invention is not limited in terms of the tissue to be treated but rather has broad application, including the resection and/or vaporization any target tissue with particular applicability to the ablation, destruction and removal of problematic joint tissues.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

In common terminology and as used herein, the term “electrode” may refer to one or more components of an electrosurgical instrument (such as an active electrode or a return electrode) or to the entire device, as in an “ablator electrode” or “cutting electrode”. Such electrosurgical devices are often interchangeably referred to herein as “probes”, “devices” or “instruments”.

The present invention is particularly concerned with the category of electrosurgical devices referred to in the art as “ablators”, i.e., electrosurgical electrodes designed primarily for the bulk removal of tissue by vaporization, though the inventive principles extend to electrosurgical device adapted for the cutting of tissue or coagulation of bleeding vessels.

Electrosurgical devices contemplated by the present invention may be fabricated in a variety of sizes and shapes to optimize performance in a particular surgical procedure. For instance, devices configured for use in small joints may be highly miniaturized while those adapted for shoulder, knee and other large joint use may need to be larger to allow high rates of tissue removable. Likewise, electrosurgical devices for use in arthroscopy, otolaryngology and similar fields may be produced with a rounded geometry, e.g., circular, cylindrical, elliptical and/or spherical, using turning and machining processes, while such geometries may not be suitable for other applications. Accordingly, the geometry (i.e., profile, perimeter, surface, area, etc.) may be square, rectangular, polygonal or have an irregular shape to suit a specific need.

The present invention makes reference to a “structural member” or “shaft” that directly conducts energy to the active electrode. The structural member is preferably comprised of elongate and rigid inner and outer concentric elements. The concentric elements may be linear or angled, and rounded, rod-like or tubular. Both the inner and outer elements are preferably conductive and more preferably formed of metal or metallic material. In certain embodiments, they may be hollow, including a lumen running therethrough that serves as a channel for the inner element or an aspiration path for removing gaseous and liquid ablation byproducts. The latter lumen flow may also serve to cool the device. However, non-lumened and non-aspirating inner element embodiments are also contemplated. In such devices, the inner element of the energy-carrying structural member may be tubular or a solid rod, with the rod cooling the active electrode by conduction of heat away from the ablating surface.

The present invention makes reference to one or more “active electrodes” or “active elements”. As used herein, the term “active electrode” refers to one or more conductive elements formed from any suitable preferably spark-resistant metal material, such as stainless steel, nickel, titanium, molybdenum, tungsten, and the like as well as combinations thereof, connected, for example via cabling disposed within the elongated proximal portion of the instrument, to a power supply, for example, an externally disposed electrosurgical generator, and capable of generating an electric field. Like the overall electrosurgical device, the size, shape and orientation of the active electrode itself and the active surface (i.e., ablating surface) defined thereby may routinely vary in accordance with the need in the art. It will be understood that certain geometries may be better suited to certain utilities. Accordingly, those skilled in the art may routinely select one shape over another in order to optimize performance for specific surgical procedures. For example, for accessing narrow structures like vertebral discs it may be desirable to use an elongated electrode of a narrow geometry, e.g., having a relatively flat profile. Thus, for the most part, choices in geometry constitute a design preference.

The profile, shape and orientation of the exposed electrically active surface(s) (i.e., ablating surface(s)) of the active electrode may likewise be optimized. The ablating surface may be elongated and/or contoured, smooth or irregular, with or without grooves or furrows, with or without an array or series of ribs, pins or other protuberance, and may incorporate apertures for the introduction of irrigant to and/or the aspiration of electrosurgery byproducts from the site.

In certain embodiments, the present invention makes reference to one or more “return electrodes”. As used herein, the term “return electrode” refers to one or more powered conductive elements to which current flows after passing from the active electrode(s) back to the electrical generator. This return electrode may be located on the ablator device or in close proximity thereto and may be formed from any suitable electrically conductive material, for example a metal material such as stainless steel, nickel, titanium, molybdenum, tungsten, aluminum and the like as well as combinations thereof. Alternatively, one or more return electrodes, referred to in the art as “dispersive pads” or “return pads”, may be positioned at a remote site on the patient's body.

In certain embodiments, the present invention makes reference to one or more “floating electrodes” or “floating potential electrodes”. As noted above, the employment of “floating electrodes” is described in detail in U.S. Pat. Nos. 7,563,261, 7,566,333, and 8,308,724, the contents of which are incorporated by reference herein. Therein, a floating potential electrode is defined as a conductive member that is not connected to any part of the power supply or power supply circuit; as such, the electrical potential of this one or more additional conductive member is not fixed, but rather is “floating” and is determined by size and position of the electrode and the electrical conductivity of the tissue and/or liquid surrounding the distal end of the device. One or more floating electrodes are typically mounted in close proximity to the active electrode and serve to concentrate the power in the vicinity of the active electrode and thereby increase the energy density in the region surrounding the active electrode. Thus, the addition of one or more floating potential electrode(s) substantially modifies the electrical field distribution, and energy deposition, in the vicinity of the active electrode without the possibility of electrode destruction since the floating electrode is not directly connected to the electrical power supply.

The present invention makes reference to “insulators”. This term is herein to refer to the non-conductive dielectric component that surrounds a distal end active electrode, covering all exposed surfaces of the active electrode with the exception of the electrically active surface (i.e., the ablating surface) that generally protrudes beyond the insulator a short distance. Accordingly, the geometry of the insulator is largely dictated by the geometry of the associated active electrode, which, as noted above, is not particularly limited. For example, the use of a substantially circular or cylindrical active electrode dictates the use of a largely tubular insulator sleeve. However, as with the overall electrosurgical device and active electrode itself, the size and shape of the insulator may routinely vary in accordance with the need in the art. It will be understood by those skilled in the art that such choices in geometry constitute a design preference and that other geometries may be used to optimize performance for specific surgical procedures.

The insulator should be fabricated from a suitable electrically non-conductive, biocompatible high temperature material. A high temperature polymeric material may be use; however, a ceramic material such as alumina, zirconia, or silicon nitride ceramic is preferred. In the context of the present invention, the word “ceramic” refers to an inorganic, nonmetallic crystalline material prepared by the action of heat and subsequent cooling. In the context of the present invention, “technical ceramics” or “engineering ceramics” are particularly preferred.

In the context of the prior art, the insulator is typically held in place by an adhesive (typically, an epoxy) and/or by a dielectric coating that covers the elongated distal element of the ablator and overlaps the proximal end of the insulator. The dielectric coating is frequently applied as a powder that is then fused to the device by curing at an elevated temperature. Alternatively, the dielectric coating may be a polymeric heat-shrink tubing. or molded in place polymer. However, the insulator may alternatively be affixed to the active electrode by means of “brazing” or “brazed joints” such as described in U.S. Patent Application Publication No. 2011/0282341 to Carmel et al.

### Utilities of the Present Invention

As noted above, the present invention is directed to electrosurgical devices and methods that employ high frequency voltage to cut, ablate and/or coagulate tissue in conductive fluid and semi-dry environments, more particularly to ablator electrodes designed for the bulk removal of tissue, particularly joint tissue, by vaporization as opposed to the simple cutting of tissue or coagulation of bleeding vessels. However, as noted previously, the present invention is not restricted to arthroscopics. Aspects are equally applicable to other uses, for example in connection with reconstructive, cosmetic, oncological, ENT, urological, gynecological, and laparascopic procedures, as well as in the context of general open surgery.

While some embodiments of the present invention are designed to operate in dry or semi-dry environments, others utilize the endogenous fluid of a “wet field” environment to transmit current to target sites. Still others require the use of an exogenous irrigant. In certain embodiments, the “irrigant” (whether native or externally applied) is heated to the boiling point, whereby thermal tissue treatment arises through direct contact with either the boiling liquid itself or steam associated therewith. This thermal treatment may include desiccation to stop bleeding (hemostasis), and/or shrinking, denaturing, or enclosing of tissues for the purpose of volumetric reduction (as in the soft palate to reduce snoring) or to prevent aberrant growth of tissue, for instance, endometrial tissue or malignant tumors. However, the present invention is not particularly limited to the treatment of any one specific disease, body part or organ or the removal of any one specific type of tissue, the components and instruments of the present invention.

Liquids (either electrically conductive or non-conductive) and gaseous irrigants, either singly or in combination may also be advantageously applied to devices for incremental vaporization of tissue. Normal saline solution may be used. Alternatively, the use of low-conductivity irrigants such as water or gaseous irrigants or a combination of the two allows increased control of the electrosurgical environment.

The electrosurgical devices of the present invention may be used in conjunction with existing diagnostic and imaging technologies, for example imaging systems including, but not limited to, MRI, CT, PET, x-ray, fluoroscopic, thermographic, photo-acoustic, ultrasonic and gamma camera and ultrasound systems. Such imaging technology may be used to monitor the introduction and operation of the instruments of the present invention. For example, existing imaging systems may be used to determine location of target tissue, to confirm accuracy of instrument positioning, to assess the degree of tissue vaporization (e.g., sufficiency of tissue removal), to determine if subsequent procedures are required (e.g., thermal treatment such as coagulation and/or cauterization of tissue adjacent to the target tissue and/or surgical site), and to assist in the traumatic removal of the device.

### Illustrative Embodiments of the Present Invention

As noted above, the present invention affords a marked simplification to construction as well as a significant reduction in manufacturing costs. Hereinafter, the present invention is described in more detail by reference to the exemplary embodiments. However, the following examples only illustrate aspects of the invention and in no way are intended to limit the scope of the present invention. As such, embodiments similar or equivalent to those described herein can be used in the practice or testing of the present invention.

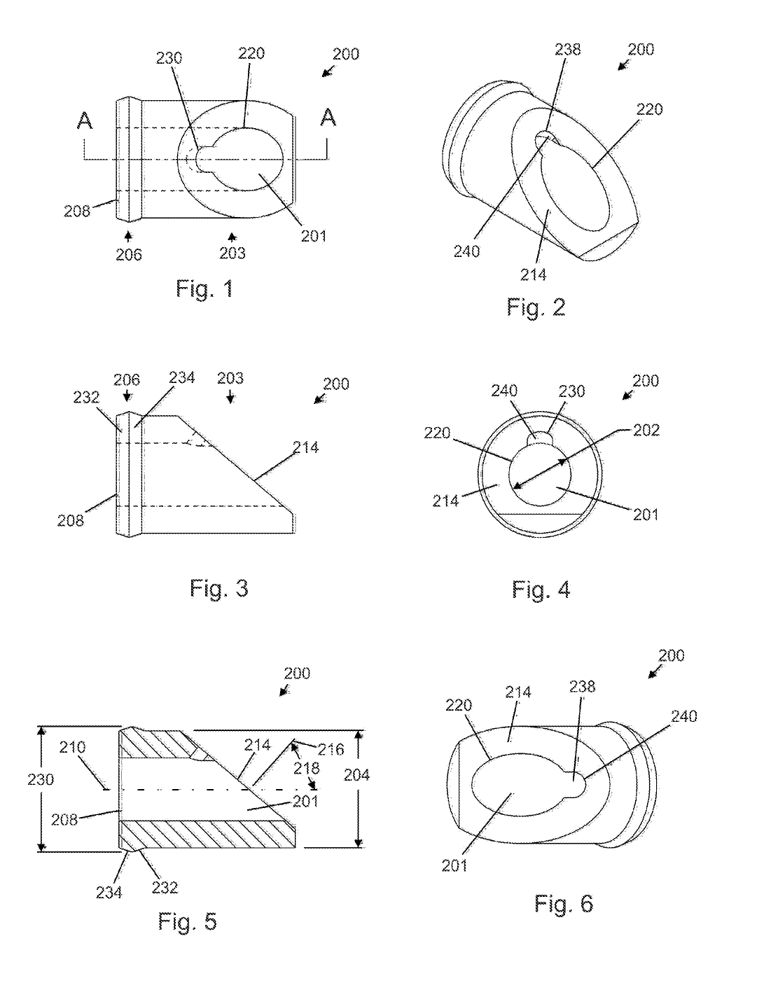

FIGS. 1 through 6 depict an insulator suitable for use in connection with an embodiment of the present invention. Insulator 200, formed from a suitable dielectric material such as, for instance, alumina, is tubular in form, has a lumen 201 with a diameter 202, and a distal portion 203 with an outside diameter 204. In this depicted embodiment, the proximal portion 206 of insulator is characterized by a planar proximal face 208 having a normal parallel to axis 210 of insulator 200. Proximal portion 206 has a maximum diameter 230, which is greater than diameter 204 of distal portion 203, and angled distal and proximal surfaces 232 and 234 respectively. Distal portion 203 has a distal end planar surface 214 having a normal 216 angularly displaced from axis 210 angle 218. Lumen 201 intersects surface 214 to form distal opening 220. At the proximal end of opening 220, recess 238 is formed, recess 238 having a proximal wall 240.

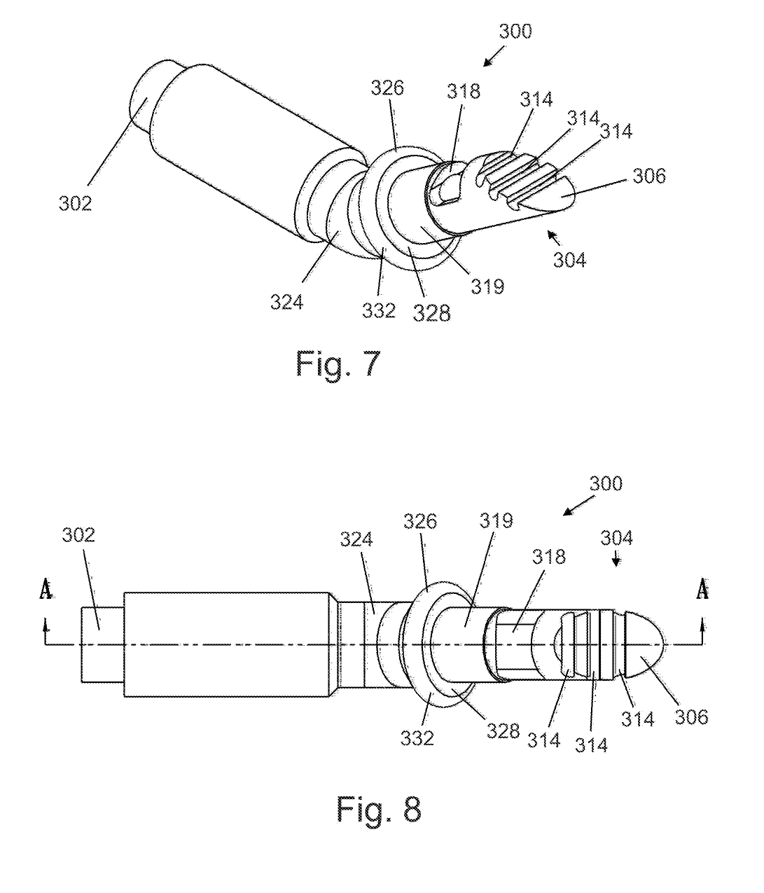

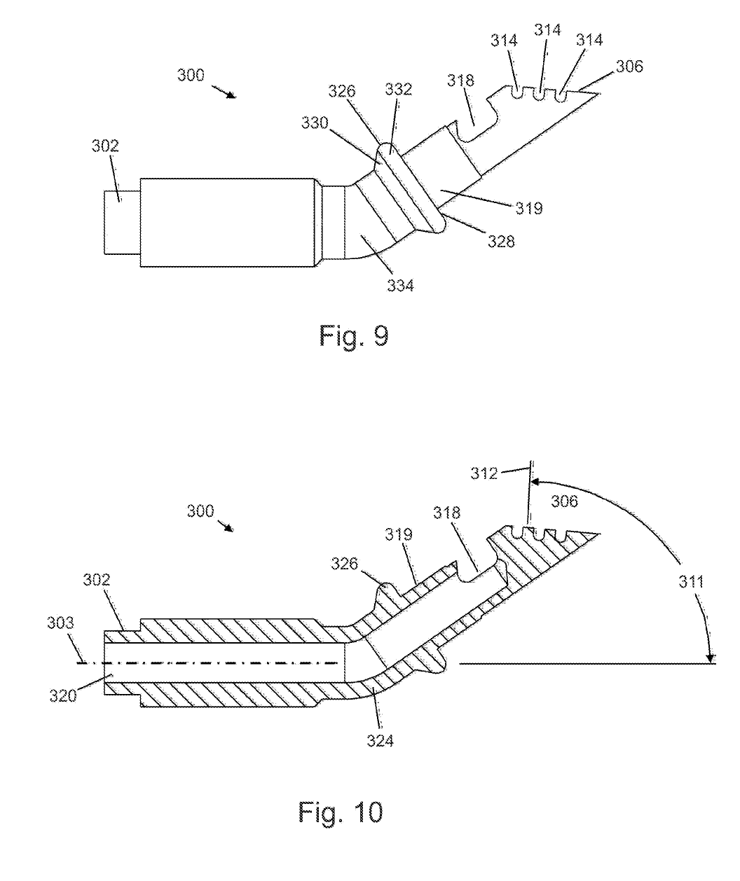

A distal end active element (or active electrode) 300 for an electrosurgical ablator formed in accordance with the principles of this invention is depicted in FIGS. 7 through 10. The construction and function of active element 300 is described in U.S. Patent Application Publication 2011/0264092 to Van Wyk, the contents of which are incorporated by reference in their entirety. As depicted herein, proximal end 302 of active element 300 is configured for mounting to the distal end of a tube. Distal end 304 has an ablating surface 306 formed thereon, surface 306 having grooves or contours 314 formed or machined therein. Just proximal to surface 306, a lateral opening—aspiration port 318—is disposed, said opening stemming from central lumen 320 of element 300. Proximal to opening 318 is tubular active element portion 319. Continuing in the proximal direction, one finds middle portion 324 of element 300, a portion having at its distal end flange 326 having a distal surface 328 perpendicular to axis 312, a conical proximal surface 330, and a radiused edge 332 disposed between distal and proximal surfaces. Normal 312 to ablating surface 306 forms angle 311 with axis 303 of element 300. Other ablating surface shapes are contemplated such as curvilinear, spherical or irregular. Ablating surfaces constructed in accordance with the principles of this invention will have at least one protuberance (e.g., a pin or raised rib) formed thereon or at least one cavity (e.g., a channel, groove, or recess) formed therein to create regions of increased current density, and the ablating surface will be positioned off-axis from the axis of the proximal portion of the active element. Active element 300 is constructed from a suitable metallic material such as, for instance, stainless steel, nickel, tungsten or titanium.

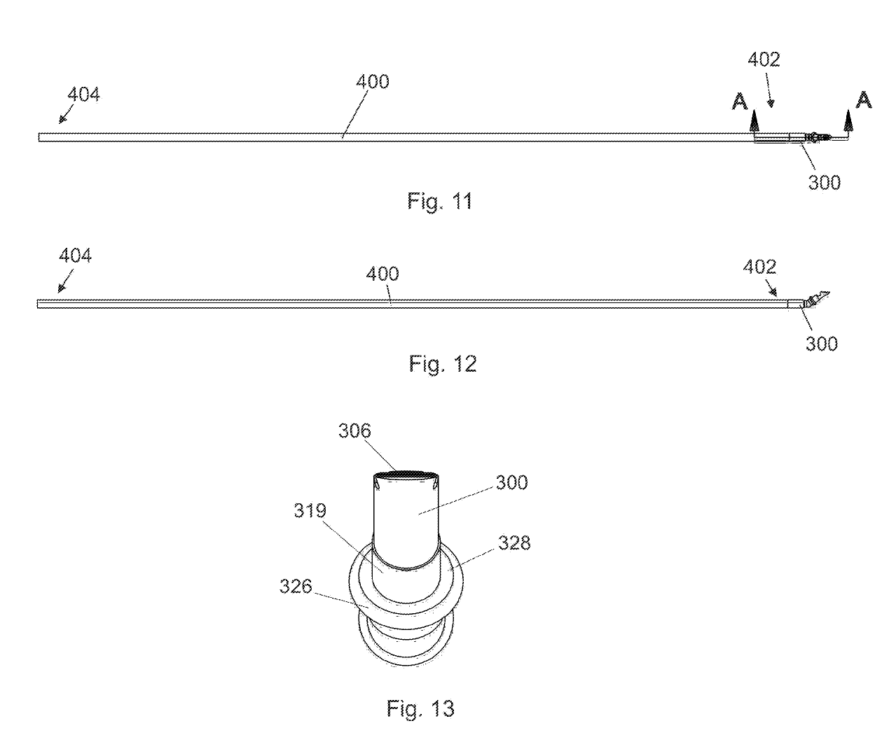

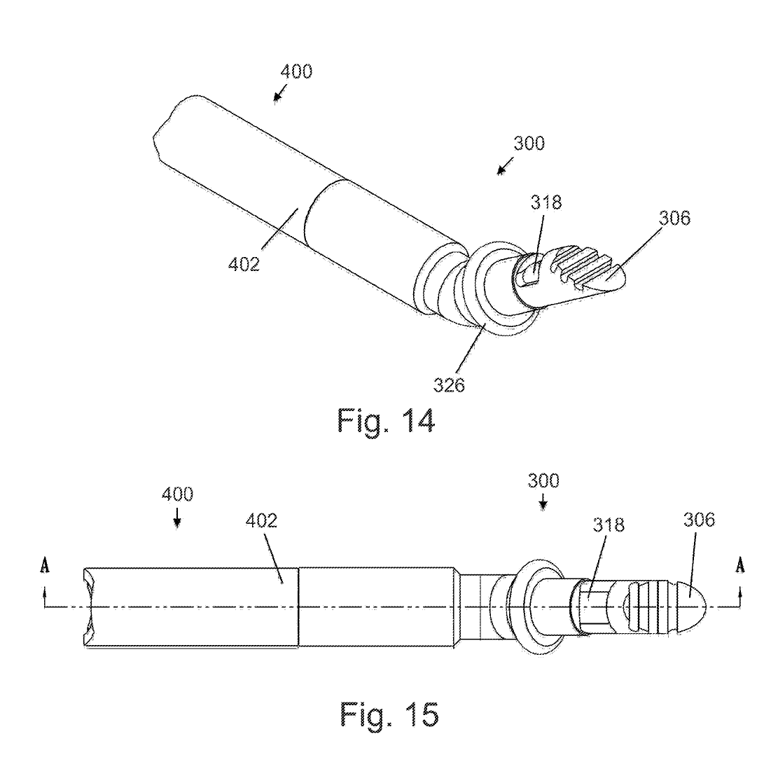

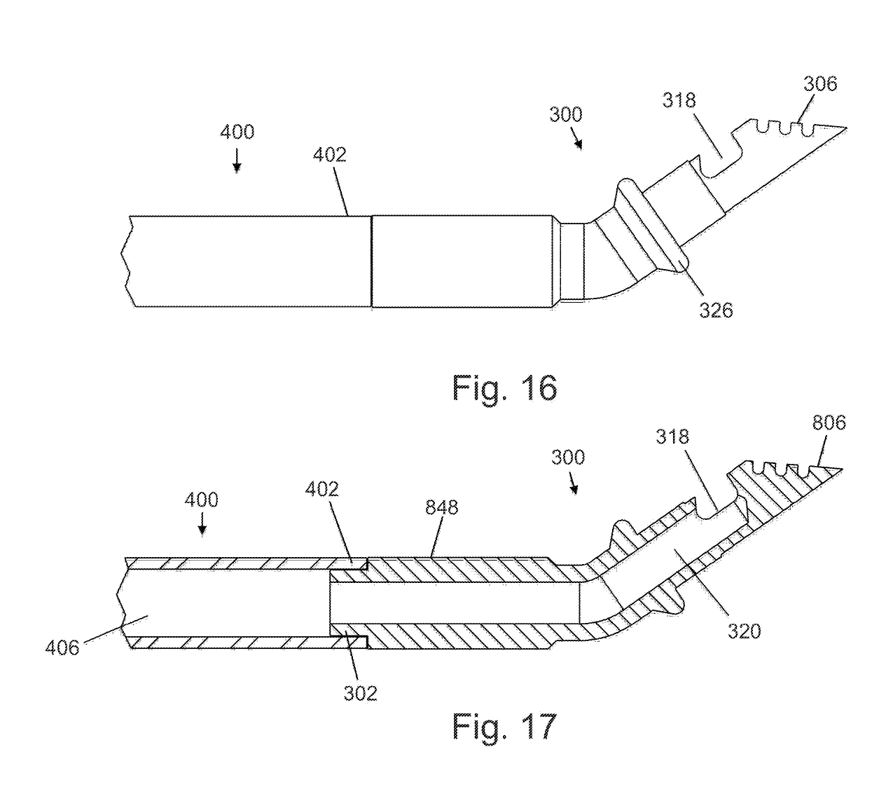

FIGS. 11 through 17 depict an assembly formed of active element 300 and tube 400, tube 400 having a distal end 402 and a proximal end 404. Tube 400 is formed from a metallic material such as 300-series stainless steel. Proximal end 302 of active element 300 is permanently affixed to distal end 402 of element 400. These joining methods include but are not limited to welding, brazing or an interference (press) fit. Aspiration port 318 and central lumen 320 of active element 300 and lumen 406 of tube 400 together form a continuous flow path.

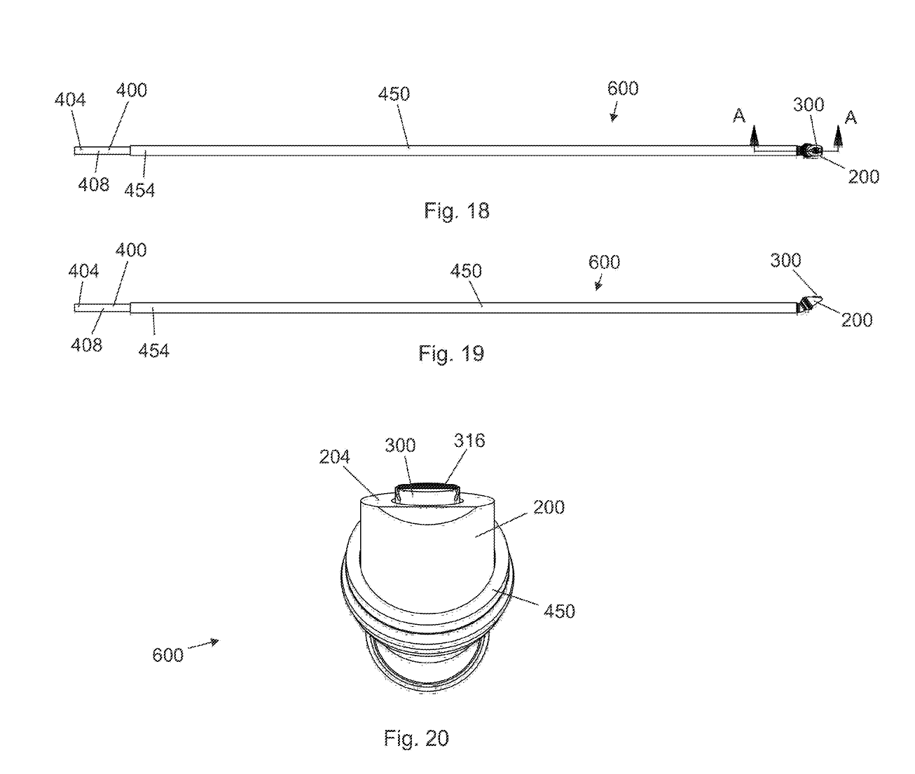

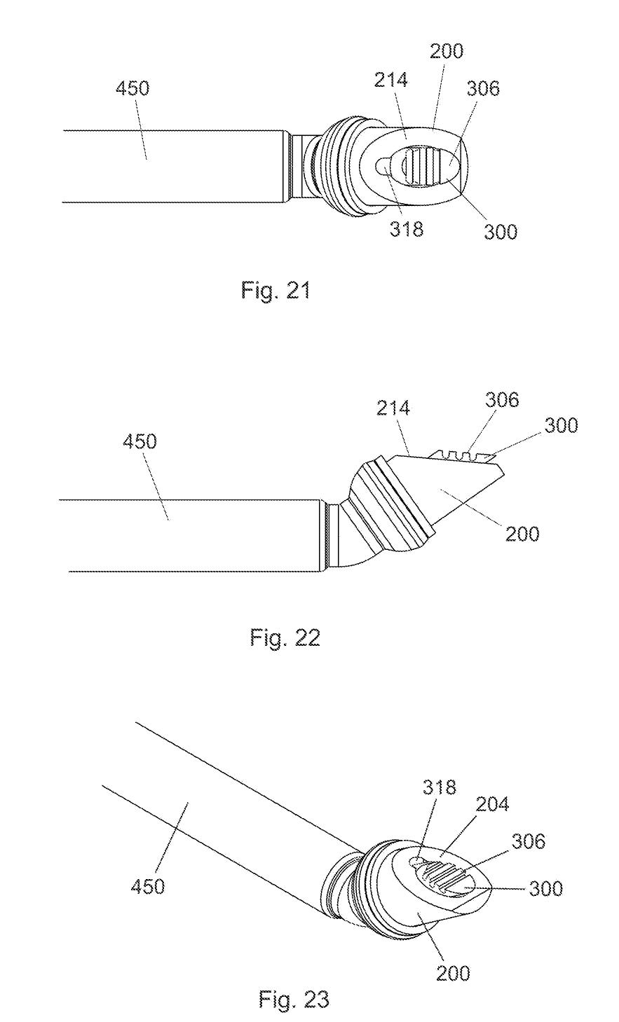

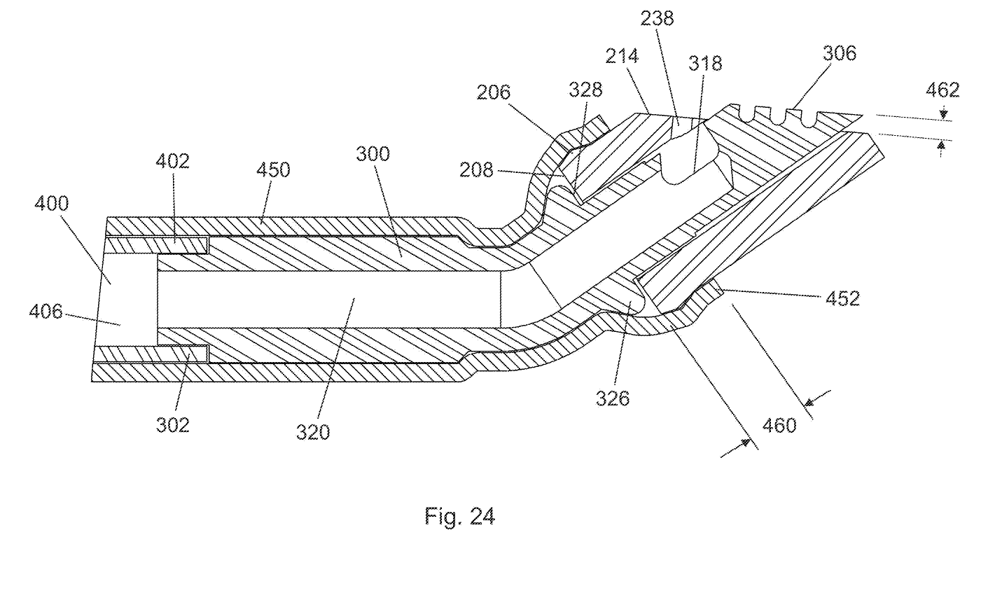

FIGS. 18 through 24 depict an insulated inner assembly 600 formed of insulator 200, active element 300, tube 400 and dielectric member 450. Insulator 200 is positioned on active element 300 with proximal surface 208 of insulator 200 positioned adjacent to distal surface 328 of flange 326 of active element 300, with lumen 201 of insulator 200 (FIGS. 1 to 6) surrounding active element 319 of active element 300, and with surface 214 of insulator 200 parallel to surface 306 of active element 300. Surface 306 of element 300 protrudes beyond surface 214 of insulator 200 distance 462. Insulator 200 may be affixed to active element 300 by brazing as described in U.S. Patent Application Publication 2011/0282341 to Carmel et al., the contents of which are incorporated by reference in their entirety. Alternatively, a suitable adhesive may be used, or mechanical means. Dielectric member 450 covers the proximal portion 206 of insulator 200, portions of electrode 300 proximal to flange 326 and tube 400 except for a portion adjacent to proximal end 404. Distal end 452 of dielectric member 450 extends distally beyond proximal surface 208 of insulator 200 distance 460. Dielectric member 450 has a proximal end 454 displaced distally from proximal end 404 of tubular member 400 to create uninsulated region 408. Recess 238 of insulator 200, aspiration port 318 and central lumen 320 of active member 300, and lumen 406 of tube 400 together form a flow path.

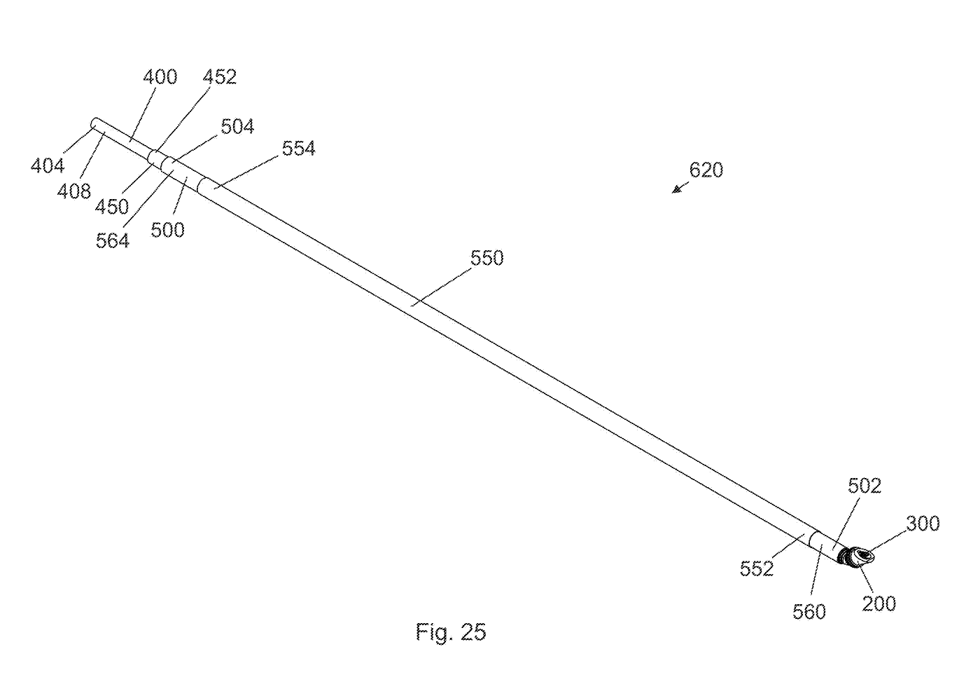

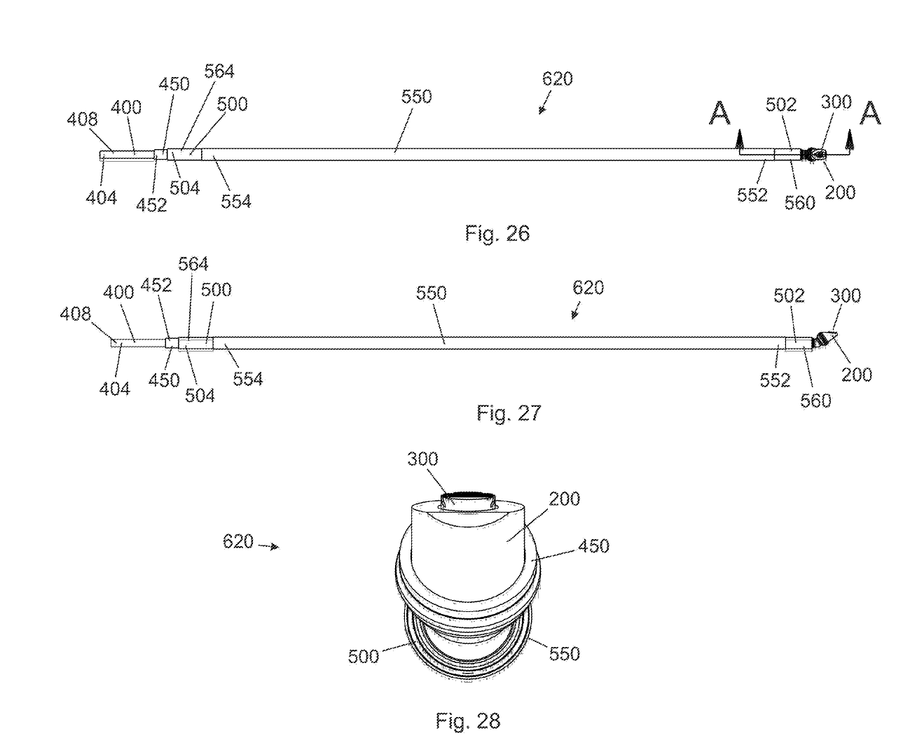

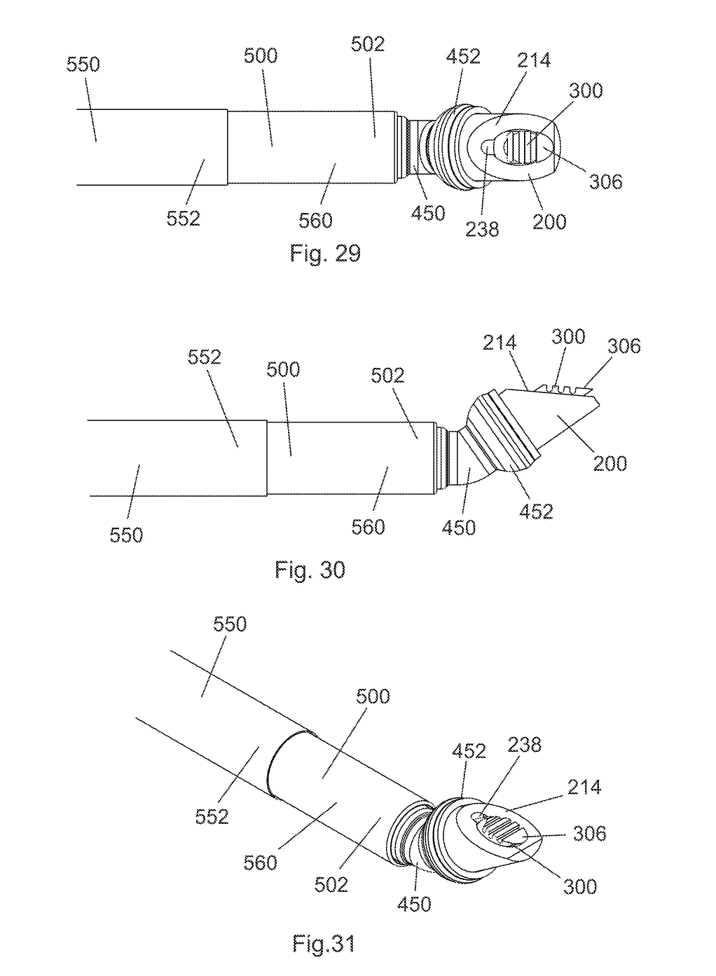

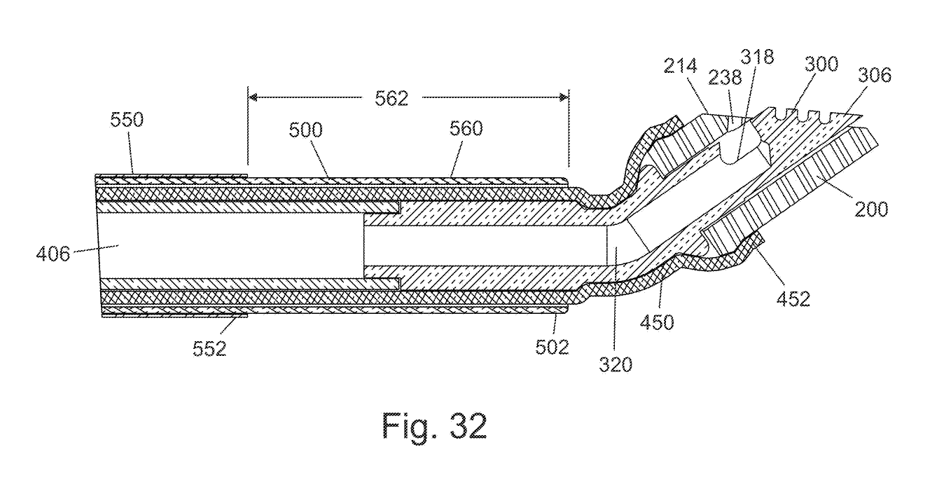

FIGS. 25 through 32 depict a distal assembly 620 for a bipolar ablator constructed in accordance with the principles of this invention. Distal assembly 620 includes insulated inner assembly 600, metallic tubular member 500, and dielectric member 550. Tubular member 500 has a distal end 502 and a proximal end 504, insulated inner assembly 600 being positioned within the central lumen of tubular member 500, distal end 502 of member 500 being proximal to distal end 452 of dielectric member 450, and proximal end 504 of tubular member being distal to proximal end 454 of dielectric member 450 such that tubular member 500 is electrically isolated from active element 300 and tubular member 400 of assembly 600 (FIGS. 18 to 24). Tubular member 500 is made from a suitable metallic material such as 300 series stainless steel. Dielectric member 550 has a distal end 552 terminating distance 562 from distal end 502 of tubular member 500 so as to form distal uninsulated region 560, and a proximal end 554 displaced distally from distal end 504 of tubular member 500 to form proximal uninsulated region 564, regions 560 and 564 being electrically connected by tubular member 500. Distal assembly 620 has an electrically conductive path formed by tubular member 400 and active element 300 between uninsulated region 408 of tubular member 400 and ablating surface 306 of element 300. Dielectric tubular element 550 is formed from a suitable polymeric material such as PTFE, Fluorinated Ethylene Propylene (FEP), Polyester, or Polyolefin.

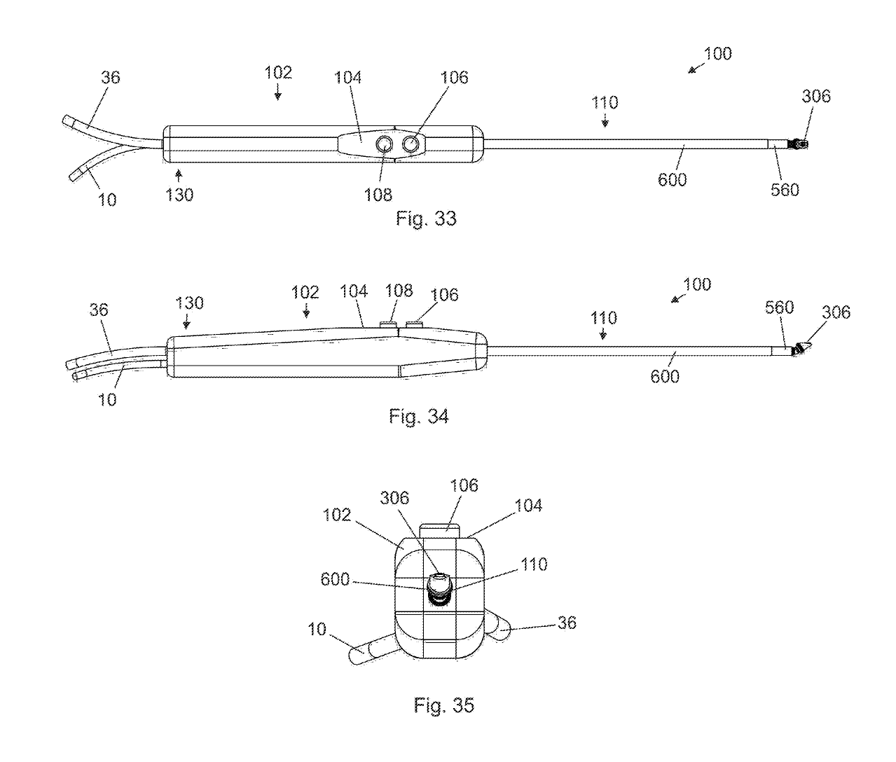

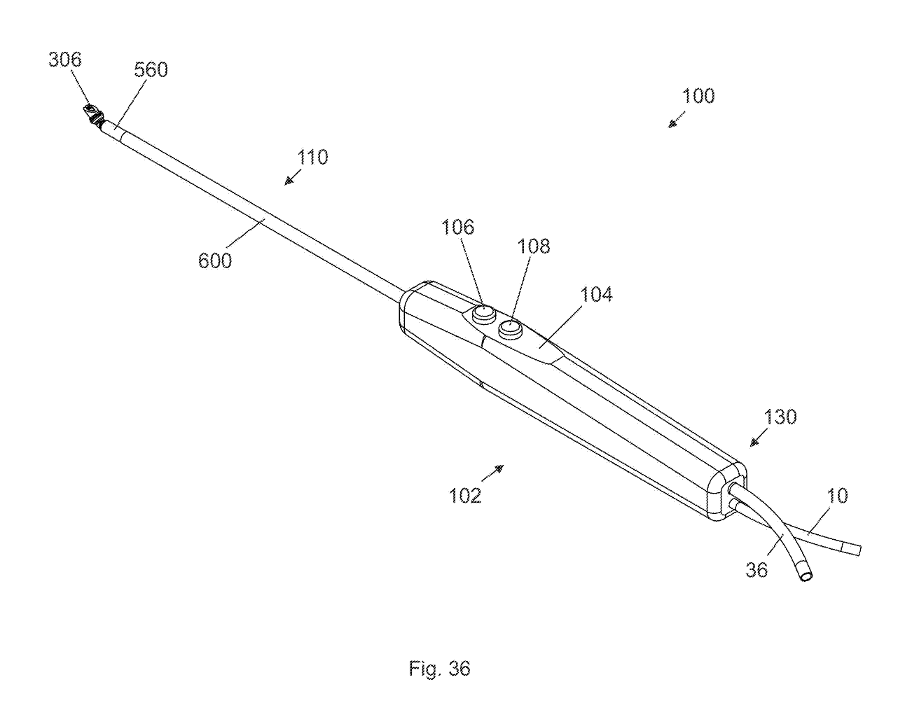

FIGS. 33 through 36 depict a bipolar electrosurgical ablator 100 constructed in accordance with the principles of this invention. Ablator 100 has a proximal portion 102 forming a handle having an upper surface 104 on which are positioned first activation button 106 and second activation button 108. Cable 10 and flexible tubing 36 pass from proximal end 130 of handle 102. Ablator 100 has an elongate distal portion 110 formed of distal assembly 600. Uninsulated region 408 of tube 400 is electrically connected via means within handle 102 to at least one conductor of cable 10, thereby creating a first conductive path from ablating surface 306 of active element 300 through tubular member 400 and means within handle 102 to at least one conductor of cable 10. Proximal uninsulated region 564 of tube 500 is electrically connected via means within handle 102 to at least one conductor of cable 10, thereby creating a second conductive path from distal uninsulated region 560 of tube 500 through tube 500 and means within handle 102 to at least one conductor of cable 10. The first and second conductive paths are electrically isolated from each other.

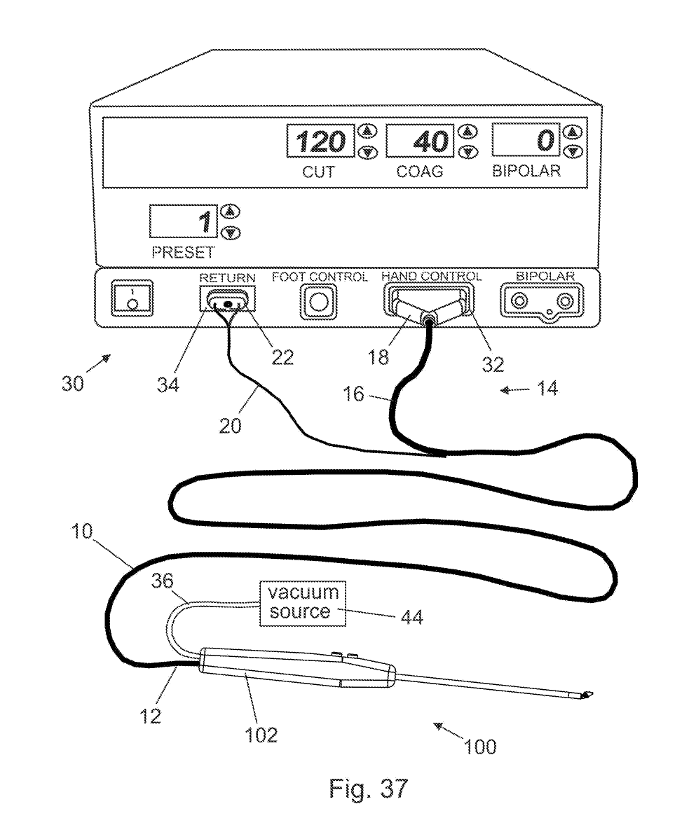

Referring now to FIG. 37 depicting an electrosurgical ablating system constructed in accordance with the principles of this invention, wherein distal end 12 of cable 10 is connected to receiving means (e.g., a recess, socket or connector) within handle 102 of device 100. Proximal portion 14 of cable 10 divides into two portions; first portion 16 having at its proximal end three-pin connector 18 configured for connection to the socket 32 for hand-controlled monopolar devices, and second portion 20 having at its proximal end connector 22 configured for connection to the socket 34 for the monopolar return. First proximal portion 16 of cable 10 provides RF energy to ablating surface 306 of active element 300 via the first conductive path previously described, and provides communication with first and second activation buttons 106 and 108 respectively such that depressing first button 106 causes RF energy of a first waveform and preset power level to be supplied to active electrode 124, and depressing second button 108 causes RF energy of a second waveform and preset power level to be supplied to active electrode 124. Second proximal portion 20 of cable 10 is connected via the second conductive path previously described to uninsulated distal region 560 of tubular member 500. Second proximal portion 20 is formed from a wire pair making connection at its proximal end by means of connector 22 and socket 34 of generator 30 to circuitry within generator 30 for monitoring resistance between the wire pair. The wire pair of second proximal portion 30 is connected via cable 20 to uninsulated proximal region 564 of tubular member 500. The return monitoring circuitry is used in monopolar electrosurgery to ensure that the connections to the return pad and the associated wires are maintained. Failure of any of these elements causes a rise in resistance sensed by the generator thereby causing the generator to display an error message and cease operation. Ablator 100 and other embodiments constructed in accordance with the principles of this invention use the same method to ensure the integrity of the return path of ablator 100 and cable 10.

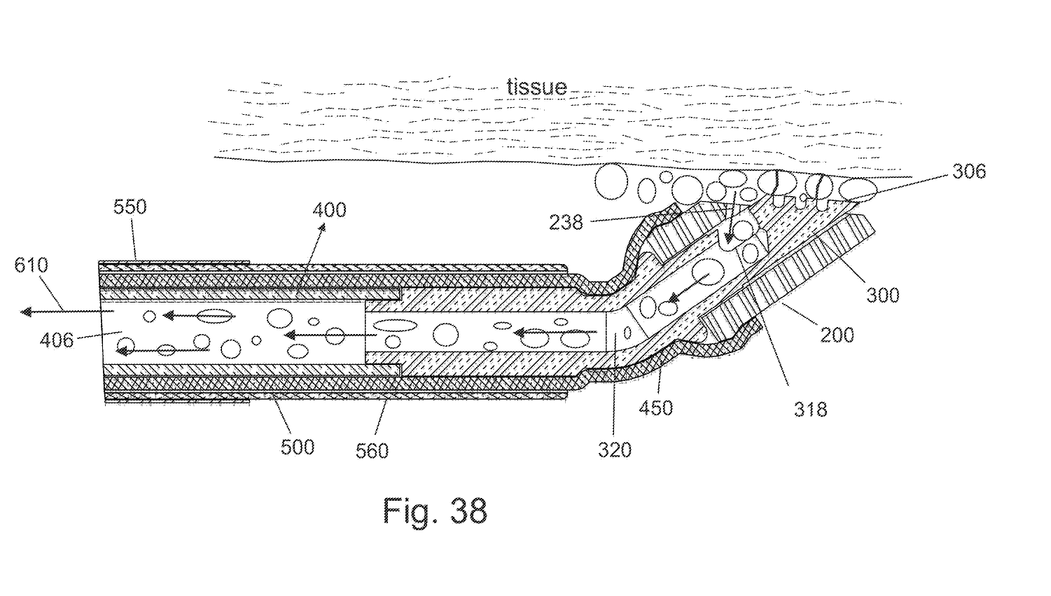

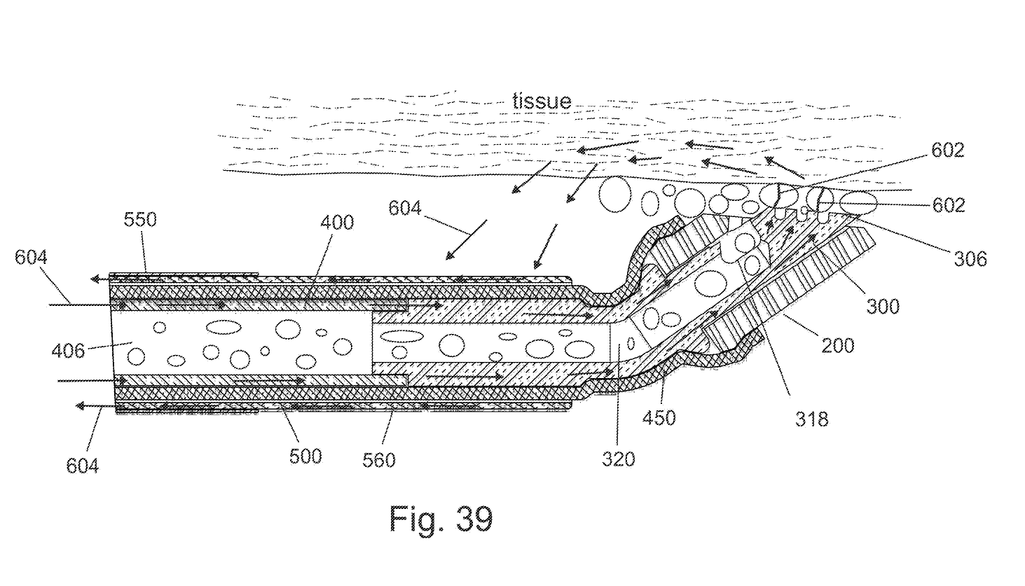

FIGS. 38 and 39 depict the distal portion of bipolar ablator 100 during use when vaporizing tissue while submerged in conductive irrigant. FIG. 38 in particular depicts the aspiration of liquid, bubbles and debris, the flow being indicated by arrows 610. Flow is from the region adjacent to ablating surface 306, through recess 238 in insulator 200, through aspiration port 318 and central lumen 320 of active element 300, through lumen 406 of tubular member 400 and therefrom via means within handle 102 of ablator 100 to flexible tube 36 and external vacuum source 44. Aspiration flow in this manner also provides cooling to element 300 and tube 400 to prevent destruction of ablator 100 due to overheating.

FIG. 39 depicts current flow during use, current flow being indicated by arrows 604. Current flows from generator 30 through cable first proximal portion 16 and cable 10 to handle 102 of ablator 100. Means within handle 102 conduct current to uninsulated proximal region 406 of tubular member 400, current then flowing through member 400 to active element 300, via arcs 602 in bubbles formed adjacent to ablating surface 306 to tissue in close proximity, and through the tissue and conductive irrigant to distal uninsulated region 560 of tube 500. Current then flows through tube 500 to proximal uninsulated region 564 of tube 500 and via means within handle 102 of bipolar ablator 100 to cable 10 to second proximal portion 20 of cable 10 and via connector 22 to the return of generator 30. Ablating surface 306 with its adjacent uninsulated portion of active element 300 functions as an active electrode while uninsulated distal region 560 of tubular member 500 serves as a return electrode. Arcs 602 vaporize tissue which they contact.

When operated in a tissue desiccation or thermal treatment mode to stop bleeding, the current path is the same except that arcs 602 are not present, with the current density at ablating surface 306 being insufficient to cause the formation of bubbles and arcs. Ablating surface 306 is placed in contact with or close proximity to the tissue to be desiccated.

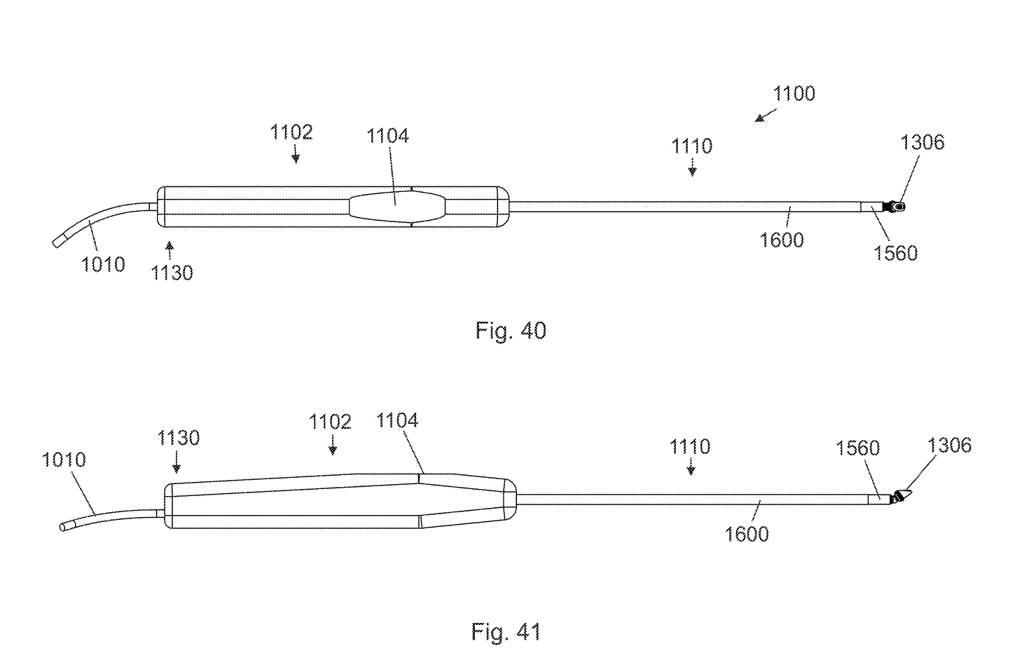

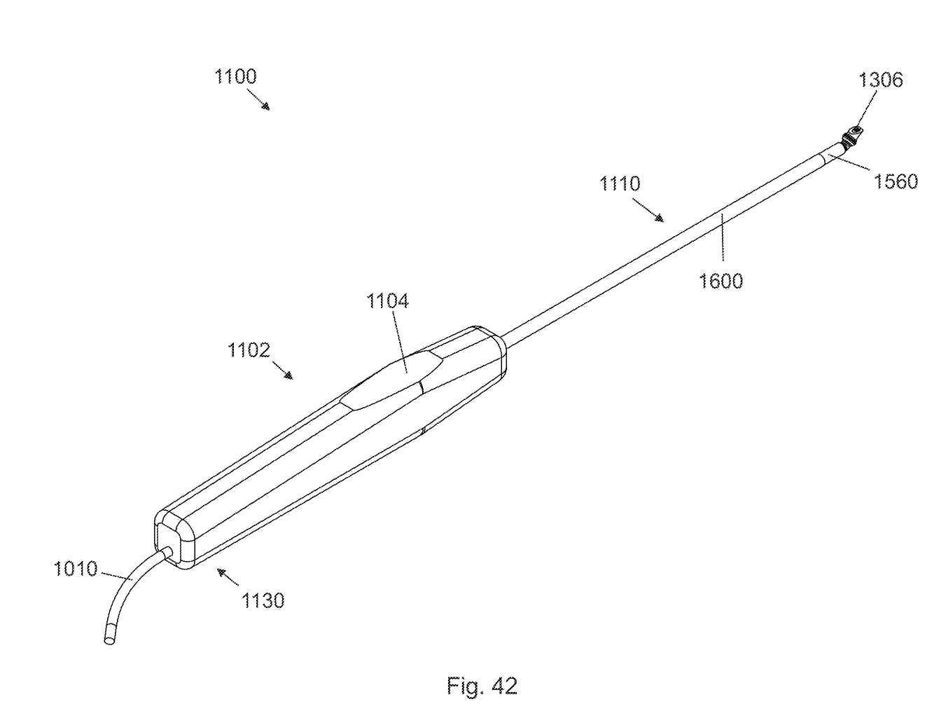

FIGS. 40 through 42 depict an alternate embodiment bipolar ablator 1100 wherein control of the device is accomplished through foot pedal controls attached to the electrosurgical generator 30. Ablator 1100 differs from ablator 100 in that first button 106 and second button 108 of ablator 100 have been eliminated, and that bubbles and debris are not aspirated from the distal region of the device during use since ablator 1100 does not provide an aspiration path. Ablator 1100 has a proximal portion 1102 forming a handle having an upper surface 1104 and a proximal end 1130 from which passes cable 1010. Ablator 1100 has an elongate distal portion 1110 having at its distal end ablating surface 1306 and distal uninsulated region 1560 acting as a return electrode.

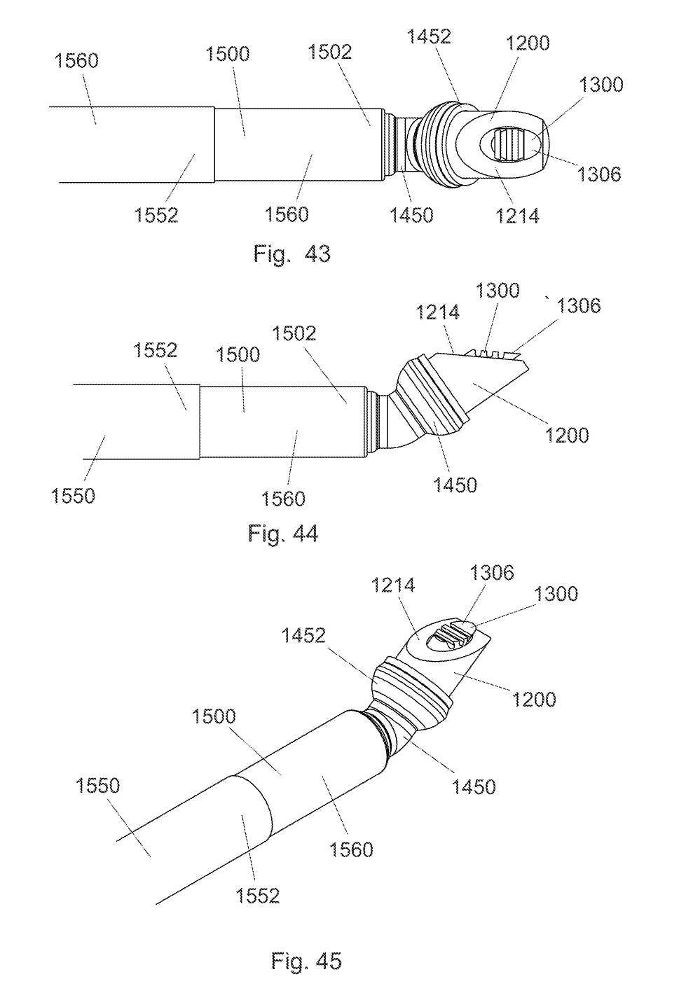

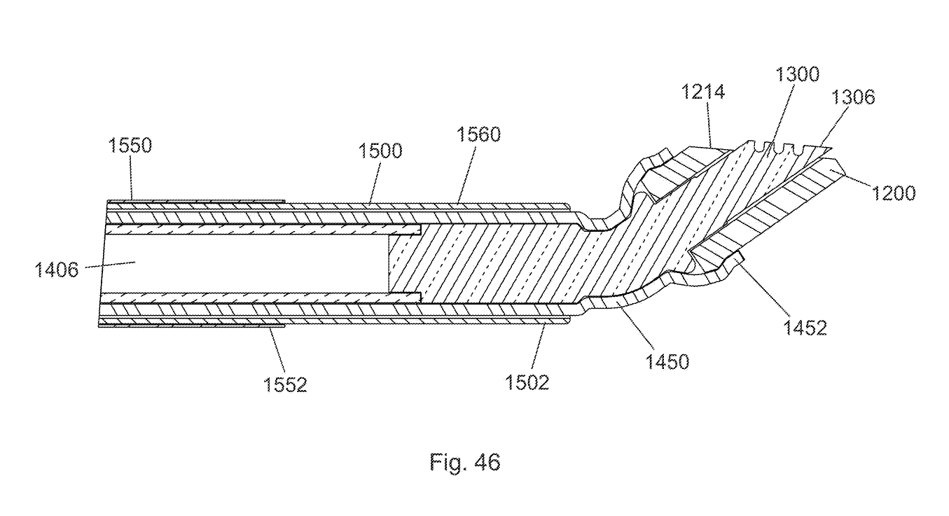

Referring now to FIGS. 43 through 46 showing the distal portion of elongate distal portion 1110 of ablator 1100, active element 1300 has a proximal portion affixed to metallic tubular member 1400 in the same manner as the previous embodiment, and a distal portion terminating in ablating surface 1306. Insulator 1200 is affixed to and aligned with the distal portion of active element 1300 in the same manner as the previous embodiment. Dielectric member 1450 covers tubular member 1400 and portions of active element 1300 proximal to insulator 1200 as well as a proximal portion of insulator 1200 in the same manner as dielectric member 450 of ablator 100. Outer tubular member 1500 and outer dielectric member 1550 are positioned in the same manner as elements 500 and 550 of ablator 100 so as to create uninsulated portion 1560. Ablating surface 1306 and uninsulated portion 1560 of outer tubular member 1500 function as an active and a return electrode respectively like ablating surface 306 and uninsulated portion 560 of ablator 100, the respective elements being connected by means within handle 1102 to cable 10 as in ablator 100.

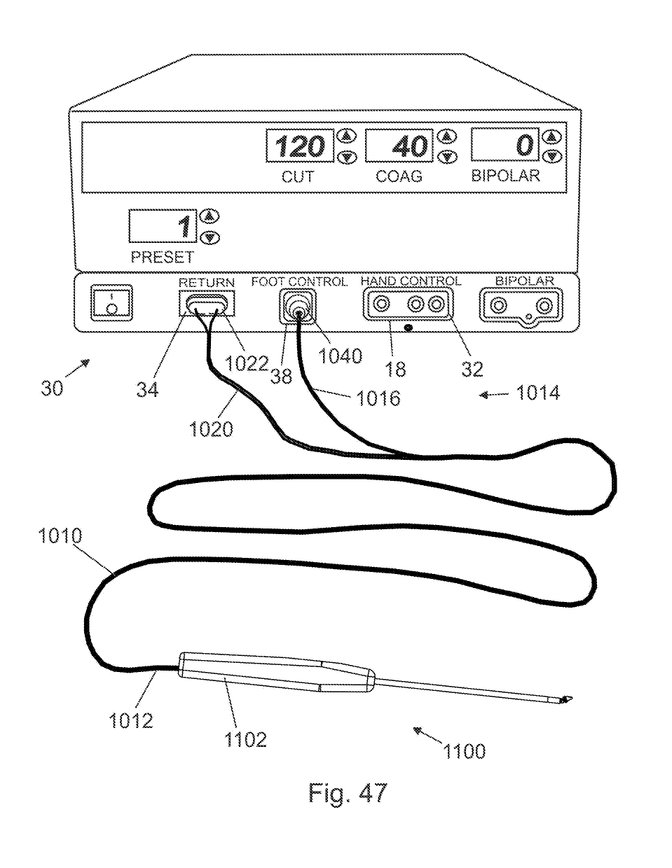

Referring to FIG. 47 depicting an ablation system for use with ablator 1102, cable 1010 has a distal end 1012 connecting to circuitry and other conductive means within handle 1102, and a proximal portion 1014 having a first portion 1016 having at its proximal end connector 1040 which is connected to the foot-control connector of electrosurgical generator 30, and a second portion 1020 with connector 1022 connected to the return connector 34 of generator 30. Activation of generator 30 when used with ablator 1100 is by foot pedals (not shown) connected to generator 30 in the standard manner. Radio frequency energy having a first preselected waveform and power level may be supplied to ablator 1100 by depressing a first foot pedal; radio frequency energy having a second preselected waveform and power level may be supplied to ablator 1100 by depressing a second foot pedal. During use, current flow of ablator 1100 is identical to that of ablator 100 depicted in FIG. 39 and the processes of vaporization or thermal treatment of tissue is identical to that of ablator 100 previously herein described. During use heat is conducted away from element 1300 by tubular member 1400.

In other non-aspirating ablators constructed in accordance with the principles of this invention, tubular member 1400 may be replaced by a solid rod to which active element 1300 is affixed at its distal end by means such as welding, brazing or mechanical fixation. So long as the member conducting power to the distal active element is a rigid metallic member positioned within and insulated from an outer metallic tubular member, and the ablating surface is oriented at an angle to the axis of the distal portion and has at least one protuberance formed on the ablating surface or at least one cavity formed therein, the bipolar ablator is within the scope of this invention.

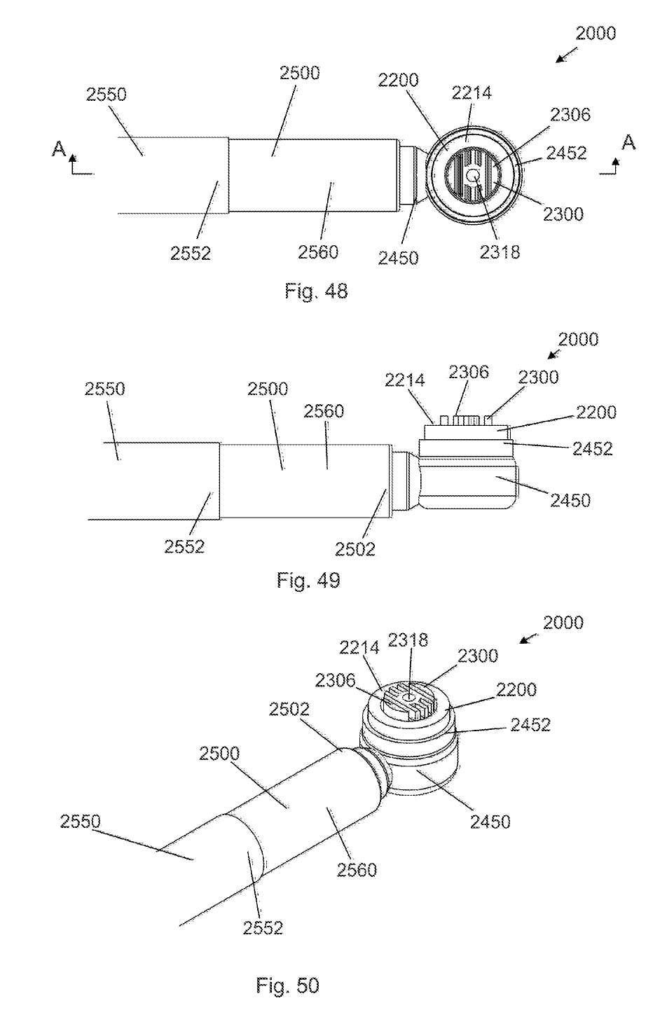

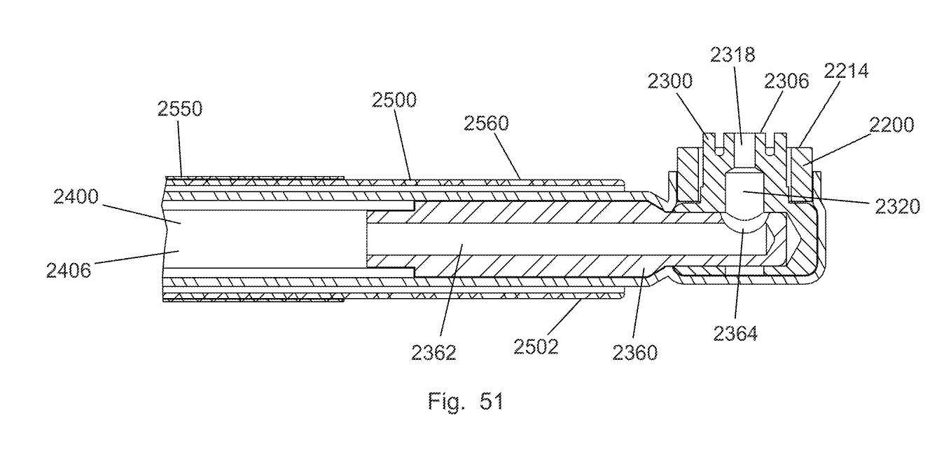

FIGS. 48 through 51 depict the distal portion 2000 of an alternate embodiment ablator identical to ablator 100 except for changes to the distal portion depicted in the figures. Ablator 100 has an active element 300 of unitary construction having a proximal portion 302 configured for mounting to tubular member 400 and a distal portion forming an ablating surface 306. Element 300 is bent to produce an ablating surface 300 oriented at a predetermined angle to axis 303 of the proximal portion of element 300. In contrast, the ablator of this alternate embodiment, the distal portion 2000 of which is depicted in the figures, has an active element that is an assembly formed of two discreet elements. The assembly together functions in the same manner as active element 300 with regard to current flow and aspiration flow. Distal active element 2300 has an ablating surface having a central aspiration port 2318 formed therein and is affixed to the distal end of element 2360, the proximal end of which is affixed to tubular element 2400. Aspiration port 2318, lumen 2320 of element 2300, passage 2364 and lumen 2362 of element 2360 and lumen 2406 of tubular member 2400 together form an aspiration path from the region distal to surface to 2306 to tubular member 2400, whereupon the aspiration path is identical to that of ablator 100 to external vacuum source 44. Current flow and aspiration flow are as depicted in FIGS. 38 and 39 except for the structural changes described and depicted.

It will be understood that aspirating devices like ablator 100 or the alternate embodiment of distal portion 2000 may be activated by either hand- or foot-controls, and may be used with a general purpose electrosurgical generator 30, or with a dedicated, special purpose generator. In the same manner, devices without aspiration may also be activated by either hand- or foot-controls, and may be used with a general purpose electrosurgical generator or a dedicated, special purpose generator.

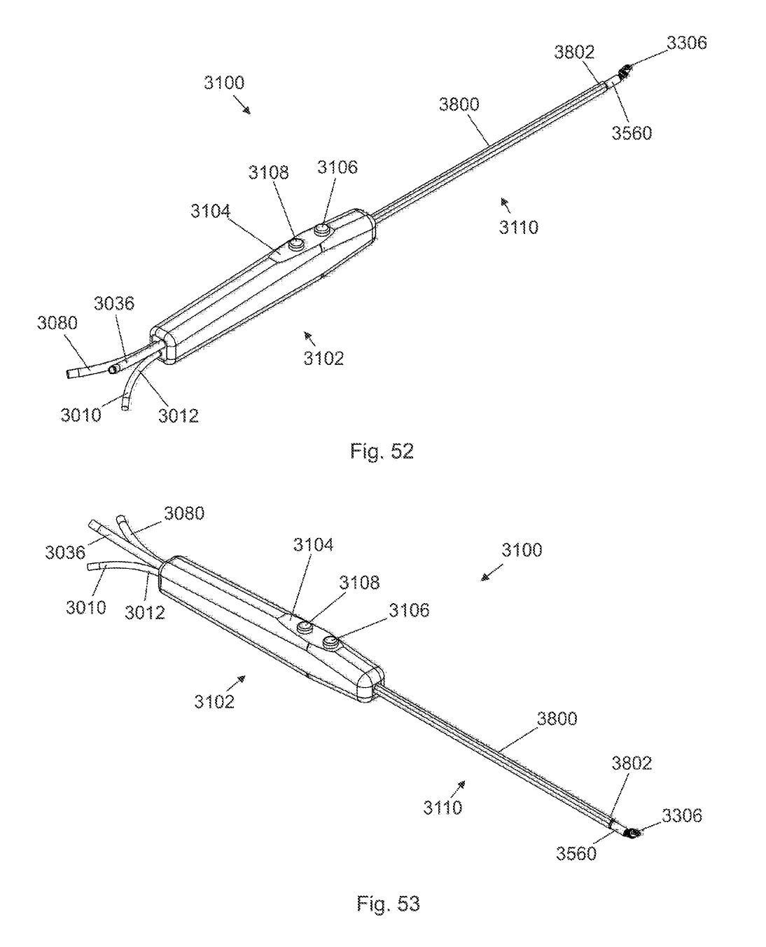

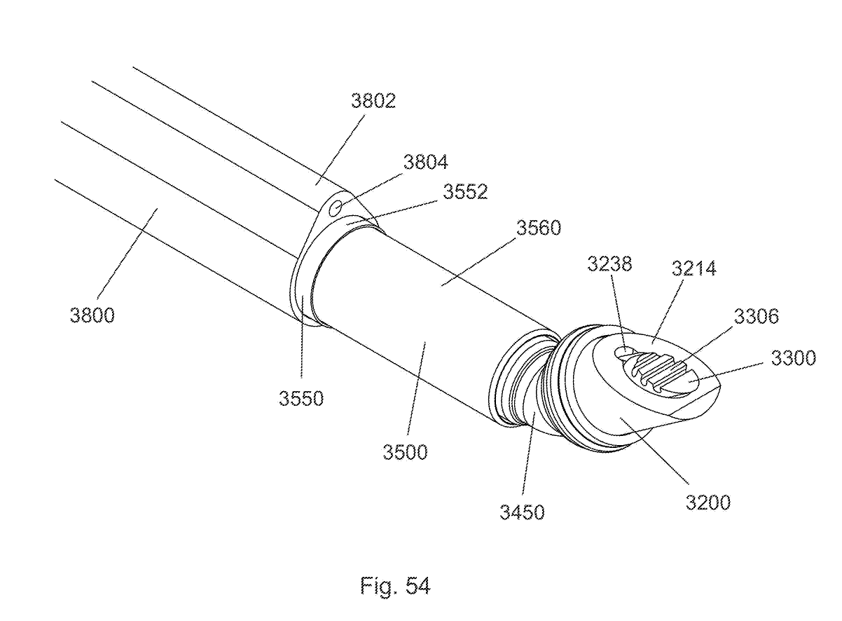

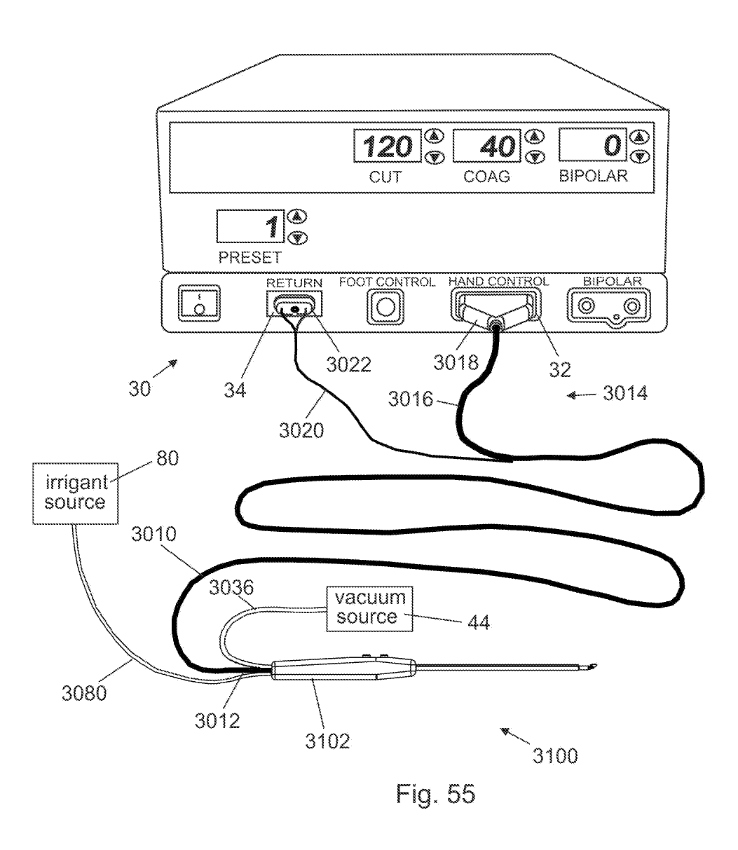

Embodiments previously herein described are intended for ablation at sites which have sufficient conductive fluids present for efficient vaporization of tissue, the fluids either coming from the body or from irrigant supplied to the site by means external to the ablation device as is the case, for example, in arthroscopic surgery. In some cases the body fluids present may be insufficient for efficient vaporization and it may be impossible to flood the site with externally applied irrigant since fluid supplied in this manner is not confined to the immediate region surrounding the active and return electrodes. In these circumstances it is desirable to supply irrigant from an outside source to this immediate region using means within the ablation device. Ablation device 3100 depicted in FIGS. 52 through 54 is identical to ablation device 100 in all aspects except for additional elements for supplying irrigant from a outside source to the region at the distal end of elongated distal portion 3110. Referring to the figures, elongate distal portion 3110 of bipolar ablator has mounted to it element 3800 terminating at its proximal end at handle 3102 and having a distal end 3802 terminating in close proximity to uninsulated region 3560 of tube 3500 which acts as a return electrode. Element 3800 is formed from a suitable polymeric material such as those which are easily extruded in the required configuration. Element 3800 has formed therein lumen 3804, lumen 3804 extending the entire length of element 3800. Proximal end 3130 of handle 3102 has passing from it flexible tubular element 3080, element 3080 being connected by means within handle 3102 to lumen 3804. FIG. 55 depicts bipolar ablation device 3100 together with other required elements of the ablation system. The system of FIG. 55 is identical to that of FIG. 37 except for the addition of external irrigant source 80 and flexible tubular element 3080 connected thereto, the distal end of element 3080 connecting to means within handle 3102 as shown in FIGS. 52 through 54. Tubular element 3080, means within handle 3102 and lumen 3804 of element 3800 together form a path for irrigant from irrigant source 80 to the distal end of elongate distal portion 3110 of ablator 3100 in close proximity to region 3560 which functions as a return electrode. Flow of irrigant from irrigant source 80 to ablator 3100 may be controlled by means well known in the art such as a roller-clamp, valve or solenoid. Indeed, functioning of the irrigant supply means of ablator 3100 is like that of other prior art devices, the irrigant forming a part of the conductive path between the active and return electrodes during operation of the device.

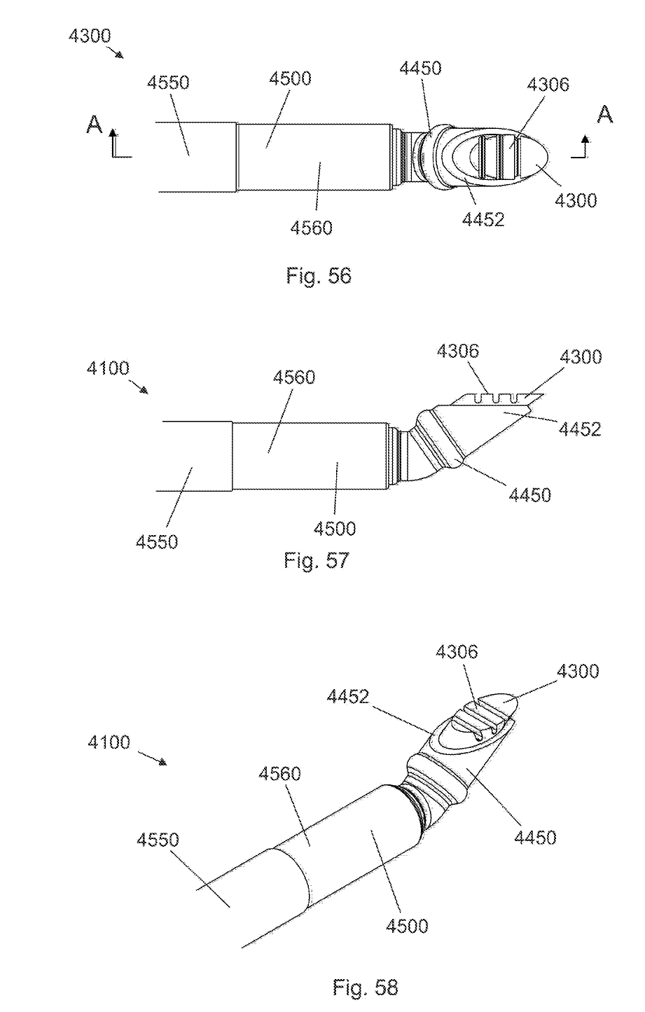

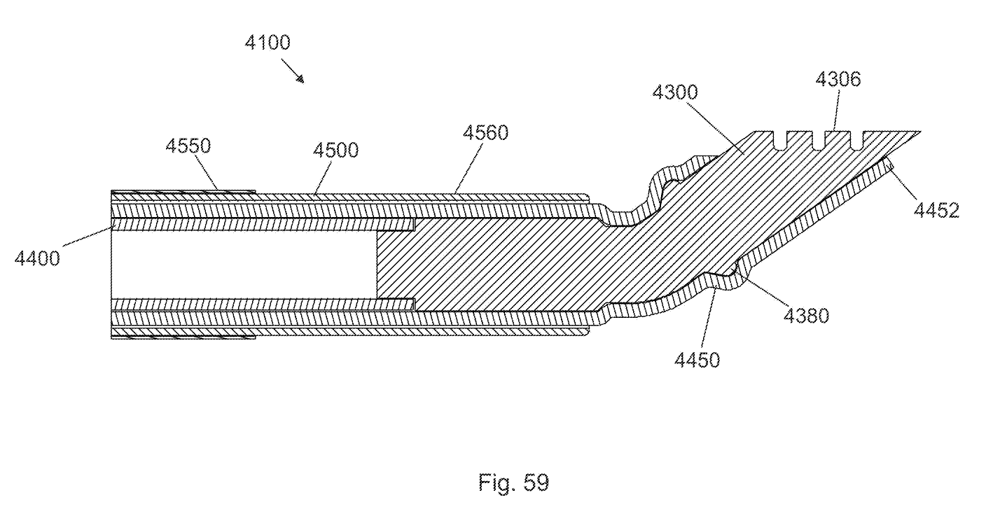

FIGS. 56 through 59 depict the distal portion 4100 of an alternate embodiment formed in accordance with the principles of this invention, this embodiment being identical to ablator 1100 depicted in FIGS. 40 through 47 in all aspects except as shown in FIGS. 56 through 59. Distal portion 4100 differs from the distal portion of ablator 1100 shown in FIGS. 41 through 46 in that insulator 1300 of ablator 1100 has been eliminated. The construction of distal portion 4100 may be suitable for applications in which ablation is achieved at low power levels and for limited durations. Referring to the figures, active element 4300 has a circumferential ridge 4380 displaced proximally from ablating surface 4306. Dielectric member 4450 extends at its distal end beyond ridge 4380, its distal end 4452 being configured such that its distal-most surface is parallel to ablating surface 4306 and displaced a distance therefrom. Ridge 4380 of active element 4300 serves to anchor dielectric member 4450 to prevent axial motion of member 4450 when the distal portion of device 4100 is inserted through the seal of a cannula or an incision in the skin of a patient. Bipolar ablators formed in this manner have a reduced cost due to elimination of the insulator, but will have a decreased usable life since the polymeric dielectric material of element 4450 will be degraded over time by high temperatures in close proximity to ablating surface 4306. However, the decreased life may be sufficient for some procedures allowing the benefit of the decreased cost. Dielectric element 4450 is preferably made from a high-temperature polymeric heat-shrink tubing material such as PTFE or PEEK. Alternatively, a high-temperature polymeric insulating material may be molded around the distal portion of active element 4300. Bipolar ablator 4100 is a non-aspirating device. The insulator on aspirating ablation devices may also be eliminated for certain applications provided that the aspiration port is positioned within the ablating surface as depicted in ablation device 2100 rather than adjacent to the ablating surface.

### INDUSTRIAL APPLICABILITY

The bipolar ablators of the present invention provide a simple construction suitable for use with a wide array of electrosurgical components and adaptable to wide range of angled positions to permit access to a variety of tissues, in an array of diverse environments so as to address a host of ablation needs. Thus, present invention maximizes efficiency and adaptability while minimizing manufacturing costs and device profile.

All patents and publications mentioned herein are incorporated by reference in their entirety. Nothing herein is to be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Such other advantages and features will become apparent from the claims filed hereafter, with the scope of such claims to be determined by their reasonable equivalents, as would be understood by those skilled in the art. Thus, the invention is defined not by the above description, but by the following claims and their equivalents.

What is claimed:

1. A bipolar ablation device comprising:
a. a proximal portion comprising a handle and proximal end configured for connection to an electrosurgical power source;
b. an elongate distal portion comprising concentric rigid inner and outer conductive elements separated by an insulating layer;
c. an active electrode formed from an electrically conductive material and in electrical communicable with said power source, said active electrode comprising a proximal section that includes a proximal end configured for attachment to the distal end of said rigid inner conductive element, a distal section that includes a distal end that forms an ablating surface, and a peripheral surface disposed therebetween;
d. an insulator formed from a non-conductive dielectric material disposed about the peripheral surface of said active electrode;
e. a return current electrode mounted to the outer conductive element in proximity to said active electrode.

2. The bipolar ablation device of claim 1, wherein said active electrode proximal section is joined to said inner conductive element by means of welding, brazing or an interference fit.

3. The bipolar ablation device of claim 1, wherein said ablating surface is off-axis relative to a longitudinal axis defined by said inner conductive element.

4. The bipolar ablation device of claim 3, wherein said active electrode proximal section is collinear with said inner conductive element while said active electrode distal section is disposed at an angle relative thereto.

5. The bipolar ablation device of claim 3, wherein said ablating surface is beveled.

6. The bipolar ablation device of claim 1, wherein said ablating surface comprises at least one protuberance or cavity that defines at least one region of increased current density.

7. The bipolar ablation device of claim 6, wherein said ablating surface comprises an array of protruding pins.

8. The bipolar ablation device of claim 6, wherein said ablating surface a plurality of raised ribs separated by grooves.

9. The bipolar ablation device of claim 1, wherein said active electrode comprises a single unity element formed from a single piece of homogenous metallic material.

10. The bipolar ablation device of claim 1, wherein said active electrode comprises an assembly of a proximal electrode component comprising said proximal end configured to mate with the distal end of said rigid inner conductive and a distal electrode component comprising said ablating surface.

11. The bipolar ablation device of claim 1, wherein said inner conductive element comprises a hollow tube.

12. The bipolar ablation device of claim 11, wherein said hollow tube comprises an aspiration lumen for removing gaseous and liquid ablation byproducts.

13. The bipolar ablation device of claim 12, wherein said active electrode comprises a cannulated tubular element characterized by an open proximal end, a closed distal end and a central lumen extending therebetween.

14. The bipolar ablation device of claim 13, wherein said active electrode further comprises a lateral opening formed in a side wall of said active electrode distal section, said opening extending through the side wall of said distal section into said central lumen.

15. The bipolar ablation device of claim 13, wherein said lateral opening is immediately adjacent to said ablating surface.

16. The bipolar ablation device of claim 1, wherein said elongate distal portion further comprises at least one floating electrode that is not directly electrically connected to said electrosurgical power source.

17. The bipolar ablation device of claim 16, wherein said return electrode and at least one floating electrode are separated by a spacing ranging from two to ten millimeters.

18. The bipolar ablation device of claim 16, wherein said return electrode and at least one floating electrode are separated by a spacing ranging from three to six millimeters.

19. The bipolar ablation device of claim 1, wherein said proximal portion is adapted to receive the proximal end of a cable that connects said handle to said electrosurgical power source.

20. The bipolar ablation device of claim 1, wherein said handle comprises first and second activation buttons.

21. The bipolar ablation device of claim 1, wherein said electrosurgical power source comprises an RF energy generator.
